(19) United States
(12) Patent Application Publication
(10) Pub. No.: US 2004/0249299 A1
(43) Pub. Date: Dec. 9, 2004

(54) Methods and systems for analysis of physiological signals

(76) Inventor: Jeffrey Lane Cobb, Ventura, CA (US)

Correspondence Address: JONES DAY, 51 Louisiana Aveue, N.W, WASHINGTON, DC, 20001-2113, US

(21) Appl. No.: 10/457,097

(22) Filed: Jun. 6, 2003

(51) Int. Cl. A61B 5/08; A61B 5/00; A61B 5/04

(52) U.S. Cl. 600/529; 600/509; 600/323

(57) ABSTRACT

Systems and methods are provided for processing and analyzing signals reflecting, physiologic processes and events in a monitored subject, especially cardio-pulmonary signals. The input signals are analyzed by in a physiological domain by creating structured data representing the physiological events reflected in the signals. Preferably, first, primitive event objects are created representing physiologically-significant portions of these input signals; second, the primitive event objects are grouped into primary event objects representing actual physiologic processes and events. Next, all objects are stored in databases, and organized in containers for efficient searching. Information may be retrieved by creating view objects which associate physiologic event objects having selected properties specified directly in physiological terms. This invention includes methods for performing the above analysis, systems and program products for carrying out these methods, and databases configured with the stored objects and views.

### 1. FIELD OF THE INVENTION

The present invention relates to improved systems and methods for processing and analyzing signals reflecting physiologic events in a monitored subject, especially signals reflecting cardio-pulmonary events.

### 2. BACKGROUND OF THE INVENTION

Currently, analysis of signals generated from ongoing physiological processes in living organisms, such as traces of lung volumes or pulse oximeter measurements, is often viewed as an exercise in traditional signal processing, and therefore employs principles that often originated in and were developed for communication and electronic technologies. Application of such communication technologies has led to progress in analyzing and interpreting physiological signals. Nevertheless, physiological signal analysis based on these standard technologies as currently practiced leaves much to be desired. One problem is that current analysis techniques tend to be inflexible; after having been designed for special analyses of specific signals, they turn out to be useful only for the specific purposes for which they were first designed. For example, one of skill in the art would typically not seek to adapt the methods and tools of an ECG analysis package designed for R-ware detection for the design of a new package for breath detection and analysis. Instead, this new problem would likely be treated de novo.

One reason for these problems may be that the concerns and goals of communications and electronic technologies have little relationship to physiological processes in living organisms. Generally, signal processing as used in communications technologies is principally concerned with constructing electronic signals that carry encoded information in limited bandwidths, then with transmitting such coded signals in more or less noisy channels also of limited bandwidths, and finally with reliably reconstructing the originally encoded information from received signals. These communication concerns and goals are at most tangentially related to an organism's physiological activities, such as breath patterns or vascular pulsations. Even when these physiological activities are electrically triggered, they have an essentially mechanical goal, such as moving air or blood in a manner controlled to maintain an organism's physical and chemical homeostasis. These activities are not used to communicate information through bandwidth-limited channels.

Therefore, the current art lacks, but is in need of, new, flexible approaches to processing signals derived from physiological processes, approaches that do not derive from and are not bound to physiologically inappropriate paradigms such as communications theories and technologies.

Citation or identification of any reference in this section or any section of this application shall not be construed to means that such reference is available as prior art to the present invention.

### 3. SUMMARY OF THE INVENTION

Accordingly, principal objects of the present invention are to overcome these deficiencies in the prior art by providing systems and methods for physiologically-motivated processing of signals measured or otherwise derived from physiological processes. These objects are achieved by systems and methods that are structured appropriately in view of the physiologic content of the signals to be processed, and that process these signals appropriately in view of the physiologic processes generating the signals. Thereby, the systems and methods of this invention use appropriate physiological paradigms and are not straight jacketed by inappropriate engineering or technological paradigms.

Systems and Methods of this Invention

Thus, instead of processing physiological signals by focusing on their communication and engineering aspects, such as their frequency spectrum, the present invention's signal processing begins with a physiological perspective. It first looks for pre-determined, primitive (or elementary) physiological events expected to occur in the input physiological signals, and then extracts characteristics of the primitive events from the input physiological signals. Primitive (or elementary) events are, for example, those physiological events that can be simply and directly recognized in an input signal, preferably from relatively short portions of an input data trace. A primitive event may be a recognizable temporal signal fragment that has a physiologically-defined meaning. In the case of lung volume measurements, primitive events might be the portions of the input signal temporally adjacent to one or more breath phases, such as the beginning of a new inspiration, the time of peak inspiratory flow, the time of peak lung volume, and so forth. Characteristics of these primitive events may include their occurrence times and their defining signal fragments along with such summary signal properties as an average, maximum, or minimum of the signal, or of its time derivative, or so forth.

However, this invention does not exclude certain time and frequency domain pre-processing. Such pre-processing may serve to filter noise and other non-physiological signals, or physiological signals that are not of interest, or other artifacts. Further, because it is often advantageous to separate signal recording from this invention's signal processing, the present systems and methods often operate on remotely-recorded, digitized, and filtered signals. In such cases, measured signals may be read from a file.

After the primitive physiological event recognition and characterization, these events are grouped or associated into the primary (or composite), basic physiological events of which they are components or elements. Primary events are preferably those events that are the basic units of physiological activity, the unit of activity that accomplish an organism's physiological goals and that are the subject of clinical or other interest. In certain embodiments, primary events may be physiologically defined in terms of, for example, measurement goals, and may be more or less granular the example events above. Since a primary event is a pattern or group of component primitive events, it may, therefore, be recognized when the proper primitive events arranged in the defining pattern or group has been found in an input signal. Representations of primary events preferably include their component primitive events along with further information characterizing the type and quality of the primary event itself. This latter information may be found from the characteristics of the component primitive events, or by comparison with the characteristics of nearby primary events, or the like. Once this physiologically-oriented signal processing is complete, the resulting structured information may be stored in persistent storage, for example, for further analysis at a later time or in a different location.

In the case of respiratory measurements, primary events are the complete breaths that actually move air for pulmonary gas exchange, and may be recognized a proper sequence of primitive inspiratory and expiratory phases recognized in input lung volume data. They are preferably represented in part as an association of the component primitive elements, and in part by the own proper characteristics, such as tidal volume, breath duration, breath rate, inspiratory and expiratory flow rates, and so forth. In the case of cardiac measurements, primary events are usually the individual heart beats that move blood, and may be recognized as patterns of primitive events found in records of thoracic or arterial pulsations or in ECG traces. Characteristics of cardiac evens may include stroke volumes, rhythm properties, rate, or so forth. For different applications, respiratory and cardiac primitive and primary events may be defined in to record other physiologic aspects of these processes.

According to further aspects of this invention, the structured information resulting from input-signal analysis, preferably including representations of primitive events with their characteristics and representations of primary events with their associated primitive events and their further characteristics, may be subject to higher-level physiologically analysis. Preferably, the input signal, either in raw or in a pre-processed form, may also be available for this analysis. This higher-level analysis examines the physiologically-structured representations created by the input signal processing in order to respond to user queries and requests, which may vary among different users. Clinical users often have interests that are different from those of users engaged in athletic training; athletic uses often have interest different from research users, and so forth. Accordingly, this invention provides structures for responding to queries seeking many different types of information, and may optionally store queries, either standard or customized for the various users. For example, the following queries: might be of interest to clinical users: show details of all apneic intervals; report the minimum, median, and maximum durations and heart rates of periods of atrial fibrillation; and so forth.

These queries are preferably specified directly in physiological terms, for example, in terms of breaths or heart beats and their characteristics, or in terms of patterns of breaths or heart beats, or the like, without reference to input signal details. Likewise, a query may generally be responded to by examining only the structured information, preferably the primary events and without reference to the input signals, for situations satisfying the physiological conditions specified by the query. However, certain queries may require reference to the input signals to determine physiological parameters not provided for in the standard construction of the structured signal representations. In some cases, query answers may be found from the details of individual primary events. These details may include the characteristics recorded for the event, the characteristics recorded for its component primitive events, and so forth. In other cases, answers may require examining sequences of primary events for particular patterns. For example, conduction defects may often be determined from examination of individual heart beat events, while arrhythmias may often only be determined from examination of patterns of sequences of heart beats. Additionally, certain respiratory conditions, such as Cheyne-Stokes respiration, also requires examination of the patterns of breath sequences.

Advantageously, this invention provides for queries that require comparison and correlation of events occurring in different physiological modalities. In embodiments where the stored information includes, for example, both cardiac and pulmonary events, concurrent breaths and heart beats may be examined to obtain more accurate answers or further answers than may be obtained from each type of information alone. For example, clinical information may be derived from heart rate variability observed during certain breath patterns, such as coughs.

Preferably, query analysis results are represented consistently with the signal analysis results as structures representing as physiological “events” of a yet more high-level or abstract character. Query results may be stored in the database for later retrieval represented as, for example, as views linking the high-level events and the primary events that are components of the abstract events. Generally, the high-level events, also referred to as “abstract” events, are groups of primary events that satisfy the physiological conditions of the query. In other cases, the high-level events may be an absence of primary events of a certain type. For example, respiratory apneas are an absence of any breath events exceeding a certain amplitude for a certain time. In any case, the associations of the component primary events along with optional information characterizing the event by type, quality, time, duration, and so forth may be associated into abstract or high-level event objects. Where the abstract events are stored in persistent storage, view structures are provided for access to these events, and may optionally include summary information characterizing the associated abstract events. For example, a user might direct an apnea query to the primary breath events recognized in an input signal obtained from a subject. This query would then find abstract apnea events representing the apneic periods in the signal and return a view representing all recognized apnea events.

In summary, therefore, this invention does not process signals measured from physiological processes merely and solely as conventional time or frequency domain data (or other similar domains). Instead, this invention recognizes at least primitive and primary physiological events in an input signal, represents these events in a structured manner, and performs further processing in a “physiological domain” of these events. Views and other stored queries are represented by a further structure which associates the lower-level events that satisfy the physiological conditions specified in the query. Further, queries may examine relationships between different physiological modalities (e.g., pulmonary and cardiac modalities) in those embodiments where data reflecting different physiological modalities are available

This invention includes not only the methods described which process input data and analyze physiologically structured representations this data, but also computer systems for performing the methods and program products for causing computer systems to carry out these methods. Importantly, the invention also includes transient and persistent computer memories with data structured according to this invention. Finally, individual aspects and sub-combinations of the elements of this invention may be separately useful and are to be included in appropriate claims. For example, input signal analysis may function alone as an individual embodiment; data analysis may function alone as a further individual embodiment; or an embodiment may include both functions acting in coordination.

The systems, analysis methods, and resulting data have numerous apparent uses. One apparent use is for medical diagnosis and treatment, which can be advanced by knowledge of the physiological state of patients and their responses to, for example, treatments. Tests of apnea and hypopnea analyses are proving the present invention to be more accurate than existing systems at machine scoring of these pulmonary events. Another apparent use is in physiological research, and it may also be useful in athletic training or in training for unusual exertion, unusual environments, and so forth.

Implementation Techniques

This invention's systems and methods are implemented using computer technologies that efficiently enable representation and manipulation of real world entities and events. Several such technologies and techniques are known. For example, in one such technology, the entities and events may be modeled according to what is known in the art as an entity-relationship model. Here, the actual events of this invention would be literally represented by structured data, such as fields, records, structures, and so forth, and relationships would be represented by links, such as pointers, addresses, or indirect references. Software, perhaps written in C, is required to explicitly create and manage and these data items. Further, these data structures and mutual pointers are preferably configured for ready persistent storage using, for example, relational database management (RDBM) systems. Such systems would typically store events of a type in a single table, and would express relationship between the stored events by keys and indices. See, for example, Date, 2000 7th ed., An Introduction to Database Systems, Addison-Wesley, Reading, Mass.

However, in preferred implementations, the structured data is further encapsulated along with functions for its manipulation in software objects. Then, use of object-oriented methods and languages automatically maintain the structured data and functions according to pre-determined specifications, known as class definitions, as well as providing structure and method inheritance and control of data visibility. The methods, syntax, and semantics of object-oriented design and programming are now well known in the art. See, for example, Coad et al., 1993, Object-Oriented Programming, Prentice Hall PTR (ISBN: 013032616X); and Yourdon, 1994, Object-Oriented System Design: An Integrated Approach, Prentice Hall PTR (ISBN: 0136363253). However, briefly and in summary fashion, object-oriented (“OO”) design is way of approaching software development that often reduces complexity and improves reliability and maintainability. With OO design, critical architecture elements describing a real-world entities or events are created as objects, which are data structures encapsulating the static characteristics of the entity, information describing the entity (its attributes), along with its dynamic characteristics, the actions of which the entity is capable (its methods). Because OO design and programming describes complex entities by a collection of encapsulated objects, these techniques promote solution of design problem by decomposition. OO design is particularly advantageous where there are strong relationships between the real-world entities being described that can easily and usefully be represented in software objects. Another advantage is that designs may be reused to describe similarly structured entities.

By way of an example, in a system useful to an automobile manufacturer, a car might be represented by an object with attributes describing the car's characteristics, for example, kind of engine, tires, body style, etc. The car object methods might include functions describing the car's actions, acceleration, braking, and the like, and describing how the car may be assembled. Further, some of the car object's attributes, the engine, tires, and so forth, might also be objects in their own right with their own more detailed characteristics and methods so that the car object would be associated with its engine object, tire objects, and other component objects. In this manner, an object oriented system can provide levels of granularity. And finally, the OO system describing cars may be reused to describe trucks with only limited modification.

Because preferred implementations of this invention are designed and implemented using these techniques, most of the subsequent description is in terms of OO techniques and implementations. Primitive events are a first type of object; primary events a further type; abstract events a third type; and views yet another type. Relations among objects of these types then expresses further aspects of the physiological processes. In addition, preferred implementation groups some or all of the signal and query analysis methods into objects. However, it should again be emphasized that this invention is not limited to OO implementations, but is fundamentally directed to analysis of physiological signals in a “physiological domain space” including structured representation of physiological events of various specificities and the analyses of such structured representations by means of further structures.

For convenience and brevity only, the following largely object-oriented descriptions use several conventions. Named object components typically represent data characteristics unless they are described as methods. Routine “getter” and “setter” methods for object elements are well known, and their descriptions are omitted. Further, visibility of object components is not specified because it is largely implementation dependent. Additionally, the literal descriptions should not be taken as limiting, because those of skill in the art will appreciate that there is considerable flexibility in constructing OO designs. For example, data and method elements may be interchangeable; particular data characteristics may be components of different objects in different implementations; and so forth. Such related implementations are intended to be part of the present invention.

### 4. BRIEF DESCRIPTION OF THE FIGURES

The present invention may be understood more fully by reference to the following detailed description of preferred embodiments, illustrative examples of specific embodiments, and the appended figures, in which:

FIG. 1 illustrates the general methods of the present invention;

FIG. 2 illustrates exemplary pulmonary signal data;

FIG. 3 illustrates a preferred state machine for pulmonary occurrence recognition;

FIG. 4 illustrates a preferred hierarchy of breath-related objects;

FIG. 5 illustrates a preferred hierarchy of view-related objects;

FIG. 6 illustrates preferred object structures in computer-readable memory; and

FIG. 7 illustrates schematically an exemplary system for practicing this invention.

### 5. DETAILED DESCRIPTION OF THE PREFERRED EMBODIMENTS

The systems and methods of this invention are capable of processing and interpreting data representing the time course of a variety of repetitive or quasi-periodic physiologic processes in a subject. For example, this invention may process arterial or venous blood pressures measured non-invasively or invasively, blood flows measured by intravascular catheters, by Doppler ultrasound techniques, etc., electrocardiographic (“ECG”) measurements of heart activity, pulmonary air flow measurements by spirometric or resistive techniques, exhaled-air composition data, intra-pleural pressure data, myographic data from, for example, intercostal muscles, and so forth. However, without limitation, the preferred embodiments described herein process cardiopulmonary, and preferably primarily pulmonary, related data produced by known non-invasive measurement techniques. Inductive plethysmography is a particularly preferred measurement technique because it may be used both for ambulatory and hospitalized subjects.

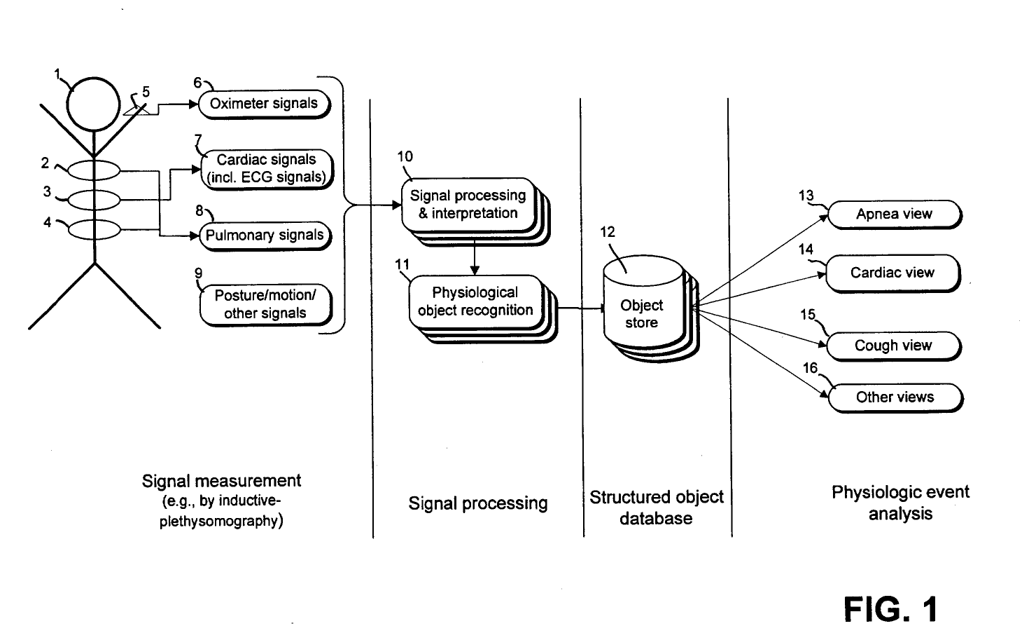

FIG. 1 generally illustrates the preferred steps by which this invention processes cardiopulmonary data. First, data signals are measured from a subject, typically by inductive plethysmography, and then directly or indirectly input to the subsequent signal processing steps. Second, the signal processing steps, after optional signal pre-processing, recognize and characterize primitive and primary physiologic objects representing input signals. The recognized physiological objects are preferably stored (that is, made persistent) in a structured object database for processing in the subsequent steps of this invention. Fourth, information in the stored objects is made available to uses by means of views that access combinations of physiological objects in response to user queries phrased in directly terms physiologic parameters of interest.

Each of these processing steps is described below, beginning with a summary of signal measurement techniques, followed by signal processing and object recognition, and concluding with physiological object analysis and views. Systems and databases of this invention are described last. The word “reflecting” is often used in the following sense: data or signals “reflecting” real physiological events means the data is related to the events so that event characteristics may be determined from the data or signals. For example, the signals may be proportional to a parameter describing the event, such as lung volume with describes breathing, or they may be monotonically related to the events, or they may be more generally related as long as the event may be determined or decoded from the signals.

### 5.2 Preferred Signal Measurement Techniques

By way of brief background, inductive plethysmography determines moment-by-moment the areas of cross-sectional planes through a subject's body, because it has been discovered that the areas of correctly-selected cross-sectional planes may provide indicia reflecting, for example, lung volumes, cardiac volumes, arterial and venous pulses, and the like. Such cross-sectional areas may be determined from the self-inductance or mutual inductance of wire loops, for example, wire loops 2, 3, and 4, placed about subject 1 (FIG. 1) in the selected cross-sectional planes (or by other inductive plethysmographic techniques). In the case of wire loops, their self-inductance depends in large part on their cross-sectional areas, and may be measured by the frequency of an oscillator including the inductance, which may then be converted to digital form. See, for example, U.S. application Ser. No. 09/836,384, filed Apr. 17, 2001 (an improved ambulatory inductive plethysmographic system) and US Pat. No. 6,047,203, issued Apr. 4, 2000 (an ambulatory inductive plethysmographic system including a sensor garment). See also, for example, U.S. Pat. No. 6,341,504, issued Jan. 29, 2002 (stretchable conductive fabric for inductive-plethysmogrpahical sensors); U.S. Pat. No. 4,807,640, issued Feb. 28, 1989 (stretchable inductive-plethysmogrpahic transducer); U.S. Pat. No. 5,331,968, issued Jul. 26, 1994 (inductive-plethysmogrpahic sensors and circuitry); U.S. Pat. No. 5,301,678, issued Apr. 12, 1994 (stretchable inductive-plethysmogrpahic transducer).

Specifically, pulmonary signals 8 may preferably be obtained from rib-cage loop 2 and abdominal loop 4. See, for example, U.S. Pat. No. 5,159,935, issued Nov. 3, 1992 (measurements of individual lung functions); U.S. Pat. No. 4,815,473, issued Mar. 28, 1989 (methods for monitoring respiration volumes); and U.S. Pat. No. 4,308,872, issued Jan. 5, 1982 (methods for monitoring respiration volumes). Raw signals 8 are filtered, smoothed, and otherwise pre-processed 10; the pre-processed signals are then combined in a calibrated manner to derive actual moment-by-moment lung volumes; and the lung volume signals are then input to object recognition processing 11. See, for example, U.S. Pat. No. 6,413,225, issued Jul. 2, 2002, U.S. Pat. No. 4,834,109, issued May 30, 1989, and U.S. Pat. No. 4,373,534, issued Feb. 15, 1983 (all methods for calibrating inductive-plethysmogrpahic breathing monitors).

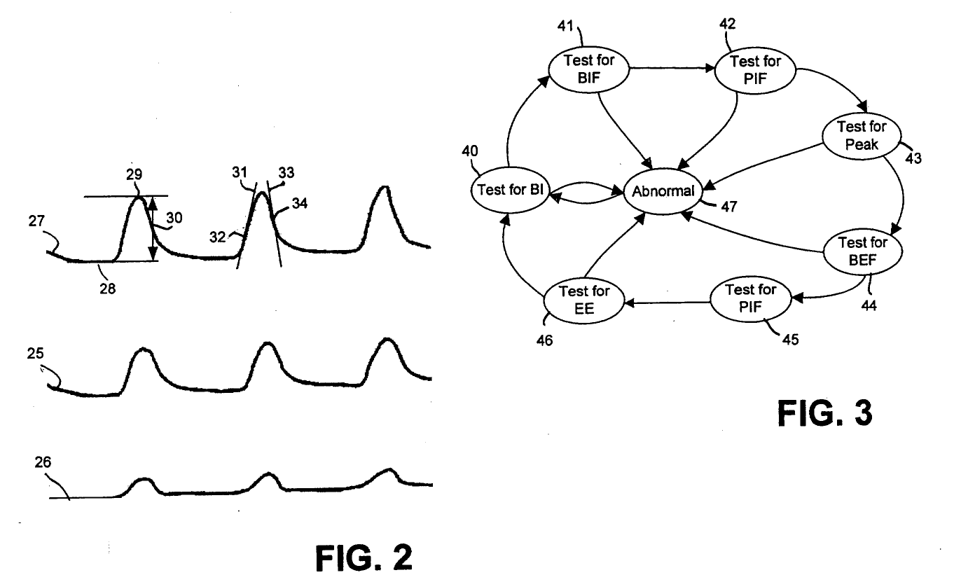

FIG. 2 illustrates exemplary inductive-plethysmographic pulmonary signals. Trace 25 is a pre-processed cross-sectional area (self-inductance) signal from rib cage loop 2; and trace 26 is a pre-processed signal from abdominal loop 4. The lung volume signal, trace 27, is a linear combination of the rib-case and abdominal signals, traces 25 and 26, with pre-determined and calibrated coefficients. Optionally, these primary signals are stored for later reference and use during object recognition 11 and analysis.

Returning to FIG. 1, cardiac signals 7 may be obtained from several sources. For example, mid-thoracic inductive-plethysmographic loop 3 provides self-inductance signals reflecting cross-sectional area in a plane through the ventricles and may be processed 10, for example, by smoothing, filtering, ECG correlation, and the like, to extract output signals reflecting moment-by-moment ventricular volume and cardiac output. Indicia of ventricular wall motion may also be obtained. See, for example, U.S. application Ser. No. 10/107,078, filed Mar. 26, 2002 (signal processing techniques for extraction of ventricular volume signal), and U.S. Pat. No. 5,178,151, issued Jan. 12, 1993 (methods for inductive-plethysmogrpahic measurement of cardiac output). Also, electrocardiogram (“ECG”) leads in electrical contact with subject 1 may provide further cardiac signals 7 which may be processed in known manners to extract, for example, heart rate, R-wave timing, and other cardiac events. Further, inductive-plethysmographic signals reflecting arterial and venous pulsations and central venous pressure may be derived from sensors (not illustrated) about the neck and limbs of subject 1. See, for example, U.S. Pat. No. 5,040,540, issued Aug. 20, 1991 (inductive-plethysmogrpahic measurement of central venous pressure); U.S. Pat. No. 4,986,277, issued Jan. 22, 1991 (inductive-plethysmogrpahic measurement of central venous pressure); U.S. Pat. No. 4,456,015, issued Jun. 26, 1984 (measurement of neck volume changes); and U.S. Pat. No. 4,452,252, issued Jun. 5, 1984 (determining cardiac parameters from neck and mouth volume measurements).

Additionally, a number of other signals may be input to the systems and methods of this invention. Signals 6 from pulse oximeter 5 may be processed by known methods to provide arterial oxygen saturation information. Other signals 9 may include, especially for ambulatory subjects, posture and motion signals from accelerometers that provide the behavioral context of concurrent cardio-pulmonary measurements. For hospitalized subjects, other signals 9 from a wide range of physiological sensors may be processed by this invention. For example, pulmonary measurements may be made in newborns as described in U.S. Pat. No. 4,860,766 (intra-pleural pressure measurements in newborns), issued Aug. 29, 1989; and U.S. Pat. No. 4,648,407, issued Mar. 10, 1987 (inductive-plethysmogrpahic determination of obstructive apneas in newborns).

Summarizing, although preferred embodiments of this invention process and interpret cardio-pulmonary measurements made by inductive-plethysmogrpahic techniques and optionally supplemented with ECG, pulse oximeter data, and accelerometer data, alternate embodiments may process any one or any combination of the signals illustrated in FIG. 1, or signals representing other physiologic processes not illustrated in FIG. 1. Further, although this invention is largely described with respect to one preferred type of inductive plethysmography, is should be understood that the systems and methods described are readily applicable to other types of inductive plethysmography. See, for example, U.S. Pat. No. 6,142,953 issued Nov. 7, 2000, or U.S. Pat. No. 5,131,399 issued Jul. 21, 1002 (both describing inductive plethysmographic techniques based on mutual inductance instead of on self-inductance.)

### 5.3 Signal Processing & Object Recognition

Briefly, step 10 may first perform such standard signal processing as is advantageous for particular signals; this processing may include smoothing, filtering, correlation, and the like. Next, steps 10 and 11 singly or cooperatively further process the signals in order to recognize and mark or annotate selected primitive physiologic events directly reflecting short portions of the pre-processed signals with physiologically-significant temporal patterns. For example, a lung volume signal may be interpreted to recognize and mark the times at which inspirations (breaths) begin, or a cardiac ECG signal may be interpreted to recognize and mark the times at which the R-wave peaks. In contrast, a primary pulmonary event usually is composed of several primitive events, and may be, for example, an entire breath, and a primary cardiac event may be an entire heart beat.

Recognizing primitive physiological events requires particular and specific physiological knowledge. An event's pattern or patterns in the particular signal being processed must be known, and preferably, the context of related signals in which the event is likely to be found. Further, primitive events to be recognized even in a single type of signal may differ in different embodiments, being chosen according to the goals of the individual embodiment. This invention encompasses alternate sets of physiologic occurrences and of significant physiologic events.

The two pieces of step 10 will now be described in more detail primarily with respect to the processing of pulmonary signals 8. It should be understood that the separation of steps 10, signal processing and primitive physiological event recognition, and 11, primary physiological object recognition, is primarily for ease of illustration and description only. In other embodiments, for example, primary event recognition may advantageously be concurrent with primary event recognition.

Pulmonary Event Recognition

In the preferred (but non-limiting) embodiment described herein, each normal breath, a primary pulmonary event, is considered to include the following six sequential primitive events: begin inspiration (“BI”); begin inspiratory flow (“BIF”), peak inspiratory flow (PIF); peak (PEAK); begin expiratory flow (“BEF”); peak expiratory flow (“PEF”); and end expiration (“EE”). In a regular-expression-like notation, a normal breath is recognized by the following pattern.

(BI. BIF . PIF. PEAK. BEF . PEF . EE)

Additional patterns may be used to recognize individual types of abnormal pulmonary events.

In turn, the primitive events may be determined from patterns of short portions of a lung volume trace. These patterns are qualitatively illustrated and physiologically defined by lung-volume trace 27 of FIG. 2 and are quantitatively described in the subsequent list. In trace 27 various primitive events are labeled on the first two of the three illustrated breaths. Thus, a breath begins at primitive event BI 28 where the lung volume is a minimum, which is indicated, for example, by the minimum horizontal line tangent to the lung volume trace at the single time 28. A breath then proceeds through the primitive BIF event (not illustrated) to the PIF event 32 illustrated for the second breath. For example, PIF occurs at time 32 at which tangent 31 to the lung volume trace has a maximum positive slope. After PIF, a breath proceeds to time 29 at which the peak lung volume (PEAK) is reached, which is also indicated, for example, by the maximum horizontal line tangent to the lung volume trace at 29. A breath then proceeds through the primitive BEF event (not illustrated) to the PEF event 34 again illustrated for the second breath. For example, PEF occurs at time 34 at which tangent 33 to the lung volume trace has a maximum negative slope. Finally, a breath is considered to end at the next lung volume minimum (not separately illustrated) which marks the (EE) primitive event.

FIG. 2 also illustrates an exemplary breath parameter known as the tidal volume. Tidal volume 30 is defined as the difference in lung volumes between the BI and the following PEAK primitive events. (Alternatively, tidal volume may be defined as the lung-volume difference between the PEAK and the following EE primitive events.) For example, tidal volume parameter may be included in the information characterizing the PEAK event.

The following list describes in more detail the characteristics of a lung volume signal defining each of these primitive events, and Table 1 presents these more specific definitions. In alternative embodiments, different identifying signal characteristics may be used to recognize events that preserve the physiological definitions above. Although the event descriptions below refer to times, it should be understood that the event preferably includes portions of the signal including the identified time that are sufficient to identify the each particular event (for example, to recognize a minimum or a maximum).

BI: This primitive event marks the beginning of the inhalation phase of a new breath. It may be determined, for example, by the following lung volume signal characteristics: as the time when either the minimum lung volume is reached; or as the time when air first measurably begins to flow into the lungs (e.g. a measurable air inflow but at a rate between 0 and 1 ml/sec, where positive flow rates signify air flow into the lungs); or as the time when the time-derivative of the lung increases beyond to zero (e.g. to between 0 and 1 ml/sec).

BIF: This primitive event marks the beginning of significant air flow into the lungs. It may be determined, for example, by the following lung volume signal characteristics: as the time when the measured air inflow first reaches or exceeds a determined threshold (e.g. 4 or more ml/sec); or as the time when the time-derivative of the lung exceeds the threshold.

PIF: This primitive event marks the maximum rate of air flow into the lungs. It may be determined, for example, by the following lung volume signal characteristics: as the time when the inspiratory air flow is a maximum; or the time when the inspiratory air flow rate first begins to decrease, where inspiratory air flow may be measured by the time-derivative of the lung volume.

PEAK: This primitive event marks maximum lung inflation during the current breath, after which the exhalation phase of the current breath begins. It may be determined, for example, by the following lung volume signal characteristics: as the time when the lung volume is greatest; or when measurable air flow out of the lungs begins (e.g. a measurable flow equal to or less than 0 to −1 ml/sec).

BEF: This primitive event marks the beginning of significant air flow out of the lungs. It may be determined, for example, by the following lung volume signal characteristics: as the time when the measured air outflow first reaches or is less than a determined threshold (e.g. −4 or more ml/sec); or as the time when the time-derivative of the lung reaches or is less than this threshold.

PEF: This primitive event marks the maximum rate of air flow out of the lungs. It may be determined, for example, by the following lung volume signal characteristics: as the time when the expiratory air flow is a minimum; or the time when the expiratory air flow rate first begins to decrease, where expiratory air flow may be measured by the time-derivative of the lung volume.

EE: This primitive event marks the ending of the exhalation phase of the current breath. It may be determined, for example, by the following lung volume signal characteristics: as the last time of minimum lung volume; as the time when measurable air flow into the lungs begins (e.g. a measurable air flow but at a rate between 0 and 1 ml/sec); or as the time when the time-derivative of the lung increases beyond to zero (e.g. to between 0 and 1 ml/sec).

Next, in simple embodiments, primitive events in the lung-volume-signal are simply recognized in the filtered signal by examining this signal's moment-by-moment amplitudes and rates of change according to the criteria defining each primitive event. If the criteria for an event are found, then that event is recognized.

In preferred embodiments, the primitive and primary pulmonary events described above are recognized in an input signal by particular methods selected from the arts of pattern classification. See generally, for example, Duda et al., 2001, Pattern Classification, John Wiley & Sons, Inc., New York. It has been found that event recognition is generally more reliable if primitive events are recognized in the context of the primary event of which they are components. Stated differently, primitive events are preferably recognized as parts of one or more patterns which define the possible primary events of which they may be parts. Such event patterns may be conveniently described by regular expressions (or similar grammatical constructs), which may be recognized by finite-state machines (“FSM”). If a primitive event is recognized which is unexpected by the patterns and their FSMs, processing then proceeds to consideration of possible signal errors or physiological abnormalities. See, for example, Duda et al., section 8.6 (Grammatical Methods). Accordingly, in presently preferred embodiments, the recognition process uses techniques based on a state machine paradigm such as the one described in the following.

However, prior to event recognition, certain signal filtering has been found to be advantageous. In particular, pulmonary signal timing may be more reliably tracked if the input signal, for example, the lung volume signal trace 27 (FIG. 2), is filtered to remove clinically insignificant lung volume variability. Generally, lung volume variability is not significant if it is approximately about 10 ml or less on a time scale of approximately about 0.5 to 1.0 sec or shorter, and an input signal is preferably filtered to damp such non-significant variability. A preferable filter thus has a moving window of approximately 0.5 to 1.0 sec, and more preferably includes 30 samples of a signal with a sample rate of approximately about 20 msec. for a window duration of approximately 600 msec. Filter coefficient may be chosen in ways known in the art so that lung volume variability below about 10 ml is damped and less than about 0.5 to 1.0 sec.

Further, once initial timings have been recognized in such a filtered signal, it is preferred to consult the unfiltered signal to refine the initial timings and to determine actual lung volume and airflow information characterizing the recognized event. For example, to refine a recognized PEAK event, the portion of the unfiltered lung volume trace between the PIF and PEF events may be more closely examined for the presence of local or global maxima. The actual PEAK may be redefined as the global maximum, or if a clear global maximum does not exist, as the average of the most prominent local maxima.

FIG. 3 illustrates an exemplary embodiment of such a FSM, having the following operation in case of a normal breath. This (virtual) machine is described as having states, at which certain actions occur, and transitions between these states. However, this invention also encompasses alternately-described, functionally-equivalent state machines, such as a machine in which actions are associated with the transitions between states, as will be recognized by one of skill in the art. Further, although it is preferred to simply recognize events in the lung volume signal, the rib cage (“RC”) and abdominal signals (“AB,”), for example, traces 25 and 26 in FIG. 2, may be also examined for occurrences of the primitive events both to confirm lung-volume-signal analysis and to determined additional information about the pulmonary events. For example, primitive events equivalent to BI, PEAK, and EE may be recognized in the signals and each signal's contribution to lung volume changes between BI and PEAK (tidal volume) determined. Also the relation between the amplitudes and phases of the lung volume, the RC, and the AB signals may be recognized. It may be significant for later analysis if all these signals were in phase and of proportional amplitude or were out of phase.

When In state 40, the exemplary FSM waits until a BI pattern indicating the beginning an a next breath is recognized in the input signal. When this event is recognized, the FSM proceeds to BIF state 41, where it waits until a BIF pattern is recognized, and, upon recognition, proceeds to PIF state 42. For a normal breath, this processing proceeds sequentially steps through the remaining primitive event components of the current breath, namely the processing proceeds from PIF state 42 to PEAK state 43, from PEAK state 43 to BEF state 44, from BEF state 44 to PEF state 45, and from PEF state 45 to EE state 46, and then back to BI to wait for the beginning of the next breath.

Table 1 presents a more detailed description of the primitive-event-signal patterns recognized in conjunction with this FSM. VCV is the volumetric change value (with units of, for example, ml/sec) and is defined as the first derivative of the lung volume signal measured over short intervals, up to approximately 200 ms. Each VCV measurement interval is preferably truncated at zero crossings and a new differentiation interval started.

TABLE 1
State Machine States

| State | Test for state |
|---|---|
| BI | The lung volume begins to increase above a threshold and VCV reaches a |
| | positive non-zero value also above a threshold. |
| BIF | The VCV measured in the input additionally-filtered signal exceeds a |
| | value of +4 starting from 0. |
| PIF | The VCV reaches a maximum positive value as confirmed by a first |
| | measurable decrease in the VCV. |
| PEAK | The lung volume reaches a maximum value as confirmed by a first |
| | measurable decrease in lung volume. |
| BEF | The VCV measured in the input additionally-filtered signal exceeds a |
| | value of −4 starting from 0 at PEAK. |
| PEF | The VCV reaches a maximum negative value as confirmed by a first |
| | measurable decrease in the VCV. |
| EE | The VCV first increases to positive value above 0, marking the beginning |
| | of the next breath. |
| ABNORMAL | Wait for input signal return to normal patterns and behavior |

Primitive event recognition depends on the current FSM state, because the FSM will recognize an event and proceed to the next state only if the recognized primitive event is the one that should follow in pattern sequence. If another primitive event is recognized, the FSM proceeds to abnormal state 47 for error processing. For example, if the FSM is in BEF state 44 and a BIF type event is next recognized, it proceeds to abnormal state 47. The FSM may also proceed to an abnormal state if the expected event is not recognized within a specified time interval, or if one or more pre-determined abnormal patterns are found in the input signal, or so forth. Once abnormal state 47 has been entered, the FSM may exit back to normal processing by, for example, testing the incoming signal for a return to normal patterns, and when the lung volume signal returns to normal, the FSM proceeds to BI state 40 to wait for the next breath. Alternately, if only a minimal abnormality was noted, state 47 might return to the next expected breath state in order to continue processing of the current breath.

In certain applications, more sophisticated recognition techniques may be advantageous. For example, Bayesian methods may be used, in which case, the FSM may be augmented or supplemented by hidden Markov models. See, generally, Duda et al., chapter 3 (Maximum Likelihood and Bayesian Parameter Estimation). Further, it may be advantageous to look ahead in the signal by, for example, recognizing at once pairs or triples (or higher order groupings) of primitive events in a longer portion of the lung volume signal. Then, the FSM states could include such pairs and triples of primitive events; conditional pair or triple recognition could present further branching possibilities. Alternately, other embodiments may represent shorter or longer portions of lung volume signals by collection of parameters which may be considered as points in a classification space. Then primitive and perhaps primary events may be recognized in this space by means of discriminant functions, either linear functions or neural network functions. See, generally, Duda et al., chapters 5 (“Linear Discriminant Functions”) and chap 6 (“Multilayer Neural Networks”).

Pulmonary Object Creation

Physiological object recognition 11 (FIG. 1) builds a hierarchy of data structures or objects representing increasingly generalized or abstracted aspects of the measured and processed input signals which is based on the primary events directly recognized in the interpreted signal by the previous processing. Although event recognition and object creation are described herein as separate and sequential steps, such a description is for convenience and clarity and is not limiting. In various embodiments, the steps may indeed be separate and sequential; in other embodiments, creation of each object may occur shortly after the recognition of the event represented.

A preferred hierarchy for most types of physiological signals includes at the lowest level objects representing primitive physiological events directly recognized in the input signals. At the next level, these primitive objects are associated or grouped into patterns by further objects representing the primary physiologic events reflected in the input signal. For example, in the case of cardiac ECG signals, the primitive event objects may represent individual P-waves, QRS-complexes, and T-waves, and the primary event objects may represent heartbeats, each of which includes its component primitive P, QRS, and T wave objects. In the case of pulmonary signals, a primary breath object may include primitive event representing the associated BI, PEAK, and EE events. Generally, primary object are first recognized 11, and subsequently additional structures are built to provide “views” of the objects stored in database 12 (FIG. 1). The views represent information useful or queried by system users.

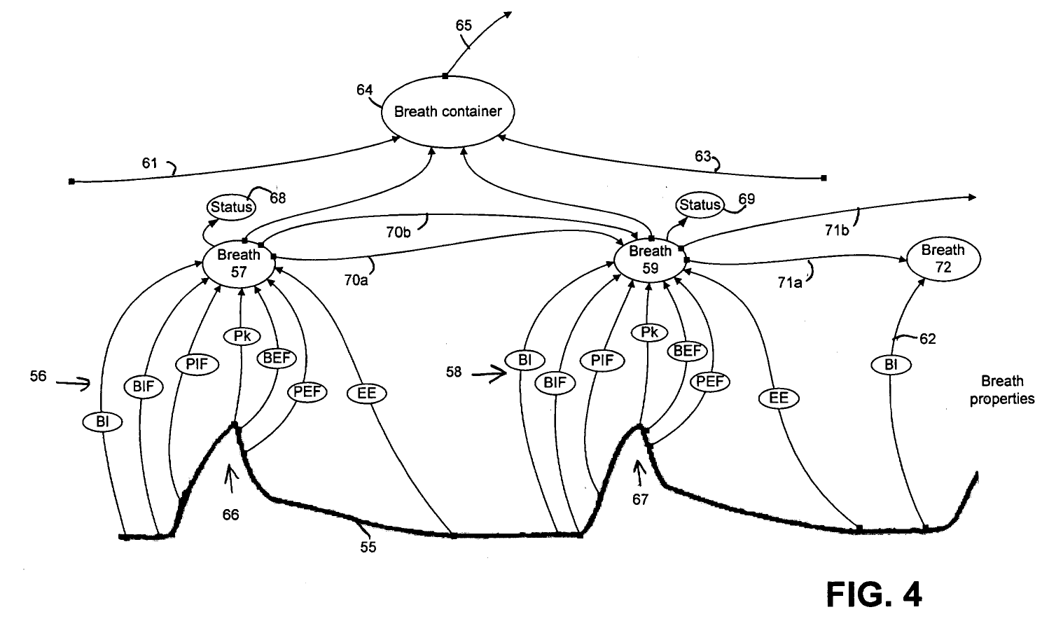

FIG. 4 illustrates a preferred hierarchy for pulmonary objects. For concreteness this figure illustrates the pulmonary objects representing lung volume signal 55, which includes two complete breaths, breaths 56 and 57, and a partially illustrated incomplete breath 72. At the first level, primitive breath event objects (also referred to herein as “breath phase objects”) are constructed, preferably one phase object for each previously-recognized primitive event. Thus FIG. 4 illustrates six primitive event objects 56—a BI event object, a BIF event object, a PIF event object; a PEAK event object; a BEF event object; a PEF event object; and an EE event object—constructed to represent breath 56, and six primitive event objects 58 to represent breath 57. Primitive event objects are instances of the class illustrated in Table 2, and preferably encapsulate, at least, the input-signal time, lung volume and air flow for the associated breath phase. For breath 72, only BI object 62 is illustrated. (As described above, routine aspects of object structures, such as “getter” and “setter” functions, constructors, and the like, are described only if they have specific structure relevant to this invention. Further, it should be understood that the object contents illustrated are not limiting. This illustrated contents generally represent what is needed for later analysis; further content may be added.)

TABLE 2
CLASS: BREATH PRIMITIVE EVENT/PHASE

| Member | Purpose |
|---|---|
| Time | Time of this primitive event in the lung volume signal |
| | (measured, for example, as the msec from the beginning of |
| | the signal file) |
| Volume | Lung volume at the time of this primitive event |
| Flow | Air flow at the time of this primitive event (measured, for |
| | example, as the VCV, or the time derivative of the lung |
| | volume) |

At a next level, breath objects represent breaths, the primary pulmonary events in this embodiment, and include at least information identifying the primitive event (phase) objects that are components of the represented breaths. Thus, breath object 57, which represents breath 66 reflected in input signal 55, is illustrated as associated with primitive event objects 56, which in turn represent the primitive events of this breath. Similarly, breath object 59 represents breath 67 by being associated to primitive event objects 58 representing this breath. Each primitive event object encapsulates at least time, value, and time derivative information from an input lung volume trace, and each primary breath object encapsulates at least information associating the primitive event components of the represented breath. Therefore, this structure may be traversed from each primary breath object to the component primitive event objects (having timing, volume, and flow data), and further to relevant portions of the input lung volume signal. Optionally, the RC and AB signals, from which the lung volume signal was derived, may also be accessed. The signal information may either be encapsulated in one of these objects (or in separate signal objects), or may be stored in a file accessible by already encapsulated timing information.

Table 3 illustrates an exemplary class, which has been found useful in the apnea/hypopnea analysis, of which breath objects are instances. Breath objects include at least information identifying the associated primitive event objects. Preferably, these objects, as illustrated, may also include further derived information useful for later analysis. The derived information may either be pre-computed and stored as object data members or computed when needed by object methods, and usually varies from embodiment to embodiment depending on user needs.

TABLE 3
CLASS: BREATH

| Member | Purpose |
|---|---|
| Associated breath | Associated breath primitive event objects |
| primitive events/phases | (either pointers or an included) |
| Time/volume/flow | Methods returning the time, lung volume, or |
| | air flow encapsulated by the primitive breath |
| | events |
| Time/volume/flow | Methods returning the differences in time, |
| difference | lung volume, or lung air flow between two |
| | primitive breath events |
| Tidal volume | Tidal volume of this breath |
| Status | Associated breath status object (an instance of |
| | the class Breath Status ) containing flags |
| | describing this breath |
| BI_next | Time begin inspiration next breath. |
| BI_next_non_{—} | Time begin inspiration next non-artifactual |
| artifactual | breath. |
| Max_OO_Phase | Method returning the maximum level of |
| | aphasic breathing recorded during this breath. |
| Min_OO_Phase | Method returning the minimum level of |
| | aphasic breathing recorded during this breath. |
| Median_expiratory_{—} | Method returning the median expiratory flow |
| flow | value for this breath. |
| Median_inspiratory_{—} | Method returning the median inspiratory flow |
| flow | value for this breath. |
| Max_Pct_RC | Method returning the percentage that the rib |
| | cage (RC) contributes to the overall tidal |
| | volume (for example, the maximum |
| | percentage achieved during a breath) (useful |
| | for detection pf coughs, sighs, etc). |
| Min_Pct_RC | Method returning the minimum percentage |
| | that the rib cage contributes to the overall |
| | tidal volume. |
| Relationships to other | Data representing the illustrated relationships |
| objects (pointers) | of an actual breath object instance with other |
| | pulmonary objects |
| Heart event data | Double-ended queue containing of pointers to |
| (optional) | heart event objects representing heart-beats |
| | occurring during the lifetime of this breath. |

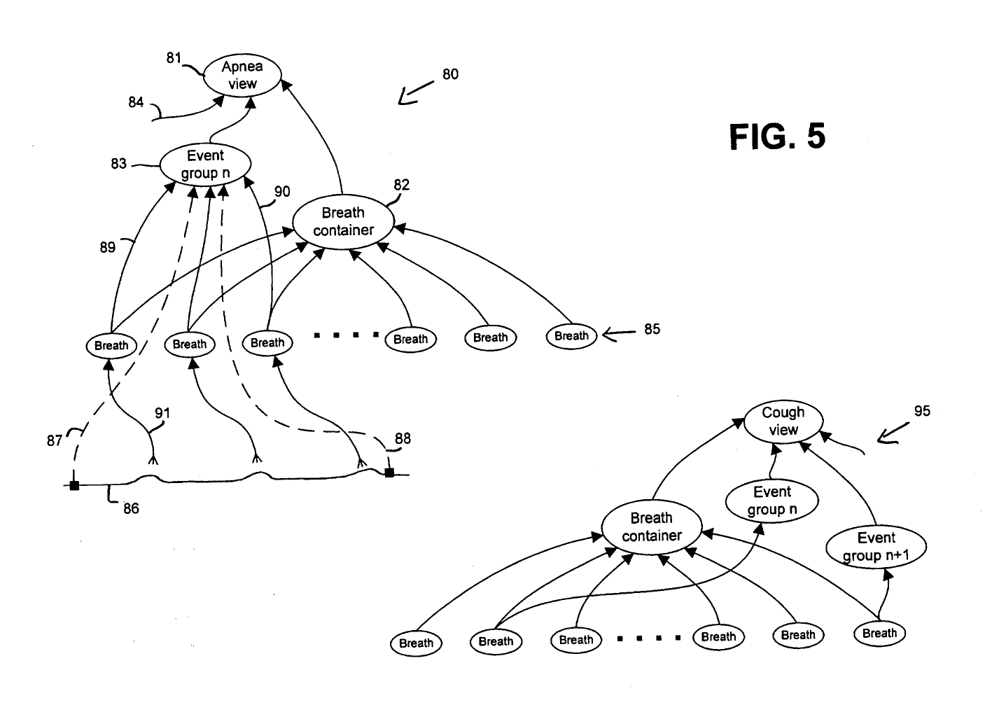

In more detail, the “time/volume/flow” and “time/volume/flow difference” methods access data encapsulated in the associated primitive event objects. (Part or all of this data may also be stored in the breath objects.). “Status” associates a breath status object, which is an instance of the class Breath Status described subsequently, containing flags describing this breath. The object data “BI_next” and “BI_next_non_artifactual” provide times of the next breath and the next non-artifactual breath, respectively. This data makes conveniently available in each breath object information concerning the gap between the ending of the represented breath, the current EE primitive event, and the beginning of the breath represented by the next breath object, its BI primitive event. In some cases, the lung volume signal during this gap is useful for finding apnea and hypopnea events. Also included is data representing the relationships of an actual breath object instance with other pulmonary objects. Exemplary relationships are illustrated in FIGS. 4 and 5. The optional “heart event data” object data is present in embodiments where heart data signals, for example from inductive plethysmographic or ECG sensors, is represented by an object hierarchy, and in such embodiments associates each breath object with temporally-coincident heart event objects. For example, if the heart event objects represent R-waves (or entire heart beats), then this data identifies the R-waves (or heart beats) that are temporally-coincident with the represented breath.

Next, the data “median_expiratory_flow” (“median_inspiratory_flow”) is the statistical median of the expiratory (inspiratory) air flow values in the input lung volume signal between PEAK and EE (BI and PEAK). This is preferably a running median with a window of approximately 1-3 min. (preferably 2 min.). This has been found useful in cough detection (especially in patients without chronic obstructive pulmonary disease (COPD)), where a cough appears as bursts of airflow scattered throughout approximately constant breathing. Max_OO_Phase and Min_OO_Phase are the running maximum and minimum of the percentage of breath intervals in which the ribcage and the abdominal contributions are out of phase. This may be simply determined as the percentage of samples during a breath at which the ribcage and the abdominal contributions to airflow are opposed to each other. Max_Pct_RC and Min_Pct_RC are similarly the running maximum and minimum percentage contribution that ribcage motions make to airflow (the remainder being the abdominal contribution). For most normal breathing, these percentages have been found to be approximately 40-50%, while in COPD patients, these percentages are in the neighborhood of 70-90% (mostly due to the emphysema component).

It is advantageous to compute and store summary status information generally concerning the breath's its quality or type during primary event recognition. This status information, preferably stored in status objects, is preliminary because it generally reflects only characteristics of and is only associated with single breaths; subsequent later-described analysis may supplement this preliminary status with the results of examining breaths in the context of past and future breaths. FIG. 4 illustrates a preferred embodiment in which separate status objects 68 and 69 are associated with their respective breath objects 57 and 59. Status objects generally contain summary data indicating whether or not the breath is normal, or abnormal by being malformed or artifactual, or apneic, or of short duration, or of small tidal volume, or so forth. Table 4 presents an exemplary class of which status objects are instances. A breath may have more than one flag set.

TABLE 4
CLASS: BREATH STATUS

| Member | Purpose |
|---|---|
| Artifact | An indication of whether or not this breath is mal-formed |
| | in some manner |
| Good | An indication of whether or not this a normally structured |
| | breath (i.e., not an artifact) |
| Apnea | An indication of whether or not this breath is possibly |
| | apneic period |
| Hypopnea | An indication of whether or not this breath is possibly |
| | hypopneic |
| Short | An indication of whether or not this breath had an |
| | unusually short duration |
| Small | An indication of whether or not this breath's tidal volume |
| | was less than approximately 50% of the baseline tidal |
| | volume for adjacent breaths |
| Sigh | An indication of whether or not this breath was a sigh |
| Cough | An indication of whether or not this breath was a cough |
| Reason | Text string providing reasons for the breath status (for |
| (optional) | example, “Breath was small because tidal volume was |
| | only 175 ml when baseline tidal volume was 400 ml”); |
| | most useful for debugging/auditing purposes |

In more detail, a non-artifactual (good) breath is one that has a tidal volume greater than approximately 50% of a baseline, is at least 1 sec. in duration, and has approximately equal inspiratory and expiratory volumes. Here, baseline tidal volumes (and other quantities) are determined as the running median or average of the tidal volumes in an approximately 1-3 min. window (preferably a 2 min. window); the window may be lagging, centered, or leading the current breath. Volumes and times are approximately equal if they are within one of two standard deviations of each other. An artifactual breath is then any breath that fails one of more of these tests. A breath may also be artifactual if it is lopsided, having inspiratory and expiratory cycles that differ by more than 200%.

Further, a flag indicating a possible apnea is set if there is at least approximately 10 seconds between the end of this breath and the next good breath and where the intervening breaths have a tidal volume of less than approximately 25% of the baseline. A flag indicating a possible hypopneic breath is set when a breath has a tidal volume of less than approximately 50% but greater than approximately 25% of the baseline. Alternatively, this flag may be set when a breath has a tidal volume less than approximately 70% of the baseline and is accompanied by a significant drop in O₂ saturation as determined from related pulse oximeter objects (see infra.). The hypopnea flag is preferably set during a later processing phase. Finally, a “short” breath is one with a duration less than approximately 1 sec. A “small” breath is one with a tidal volume less than approximately 50% of the baseline.

Preferred embodiments also include breath container objects designed to simplify breath-object access. Generally, container objects associate a number of breaths that are related by convenient criteria. For example, one container may associate all the breath objects recognized in a single input data file; another container may associate all breath objects recognized for a particular subject from input data recorded on particular dates; and so forth. Thus, FIG. 4 illustrates breath container object 64 which associates breath objects 57 and 59 as well as previous breath objects 61 and following breath objects 63. Also container objects may be associated for various purposes. For example, if the breath objects recognized in a single input data file from a particular subject are associated in a single container, a further container object may associate all such container objects for that subject. Thus, association 65 relates container object 64 to a further container object or other object.

Table 5 presents an exemplary class of which container objects are instances.

TABLE 5
CLASS: BREATH CONTAINER

| Member | Purpose |
|---|---|
| Breath event data | Double ended queue of breath event objects. |
| Find breath event | Methods for searching this breath array and |
| | returning one or more located breath objects. |

Object instances of this class include data associating a number of breath objects. They also include search methods for access the associated breaths. These methods might find the next breath, find the previous breath, find the first breath after a certain time, find breaths with certain characterizing data, and so on. In embodiments also including objects representing other physiological processes, breath containers may also be indirectly related to, for example, heart containers.

Object Creation Structures

In preferred embodiments, the methods, functions, and procedures which recognize input signals and create the above-described objects are structured as instances of an recognition and creation class presented in Table 6.

TABLE 6
Class: Signal Recognition and Object Creation

| Member | Purpose |
|---|---|
| Breath container | Current breath container being populated |
| Build_Cache | Methods for data acquisition, such as filling |
| | internal buffers from external data sources |
| Filter | Methods for performing filtering, including a |
| | running average filter on data being processed |
| Flow | Methods for performing time derivative of lung |
| | volume to find air flows |
| State machine | Methods for recognizing objects in input data, |
| | preferably including creation of recognized |
| | primitive and primary objects |
| Breath_Score | Method for returning the breath-by-breath status |
| | objects, including the calculation of breath-by- |
| | breath baseline breath volume |
| Persistence methods | Methods for object persistence and for marshalling/ |
| | de-marshalling the data to/from a file stream for |
| | export/import. |
| Logging/reporting | Methods for performing audits on the breath |
| | detection process, including simple reporting on |
| | the analysis results, such as the number and |
| | frequency of each breath type (apnea, cough, etc.); |
| | primarily for auditing and debugging. |

Here, the “breath container” data points to or associates the object instance of the breath container class currently being populated from the processing of an input data stream. The “filter” and “flow” methods perform various filtering and time differentiation operations on the input data in order to return data for use by event recognition methods. Event recognition is performed by methods labeled “state machine.” These methods execute the FSM, or other recognition engine, on the filtered input data in order to recognize primitive and primary events and also construct and initialize their corresponding, representative objects. After a primary breath object has been recognized and constructed, the “breath_score” method examines this object and constructs and initializes a corresponding status object. The persistence member methods manage persistent storage and retrieval of created objects, and optionally also provide for export of objects to a file for transfer to another system and import of objects from a file created by another system. Next, the “logging/reporting” methods are auditing and debugging tools.

These methods also build links between each breath and the next sequential breath whether it is a good (non-artifactual) breath or not, and further links between each breath and the next sequential good breath. For example, in FIG. 4, since breaths 57 and 59 are both taken as good breaths, link 70a from breath 57 to the next sequential breath of any type points to breath 59, as does the link, link 70b, to the next sequential good breath. However, the next sequential breath after breath 59, breath 72, is assumed to be either not good or an artifact. Therefore, although link 71a from breath 59 to the next sequential breath of any type points to breath 72; the link to the next sequential good, non-artifactual breath, link 71b, points beyond this breath to a later breath.

Finally, “build cache” methods interface to input data sources and deliver input data to the other processing functions thereby insulating them from details of these data sources. In a preferred embodiment, the build cache methods obtain data from the following data-producer classes:

Data source class—a super-class defining reading time, volume, and other data from generic data files;

File source class—a derived class that reads data from a processed file (such as an export/import file);

Raw source class—a derived class that reads data from a general unprocessed data source and may include source specific data smoothing and buffering (including time-centered filtering); and

Live source class—a derived class that reads signals directly from a data sensor and may also include source specific data smoothing and buffering

An implementation of this invention has at least one and may have more than one object instance of this class active when an input data stream or file is being processed.

Object Database

The objects recognized and created during input signal processing are data used for later user analysis. Therefore, although they may be maintained in main memory, it is preferred that they be stored in persistent storage as database 12 (FIG. 1). Thus, the persistence methods for persistent storage and retrieval of objects, and may also provide for marshalling/de-marshalling objects between memory and files for external transfer. With such an object database, later uses and analyses may be configured as database queries returning data. Optionally access to the returned data may be made persistent so that the queries are analogous to the SQL “view” concept.

Database 12 may be an object-oriented database system capable of directly accepting created physiological objects. Alternately, this database may be, for example, a relational database management systems (RDBMS), in which case a further layer of software is required to provide object oriented interfaces to database 12. Such software would marshal/de-marshal objects between an object format in-memory and a relational table structure in the database

Commercial RDBMSs that can be used in this invention include Oracle 9i, Microsoft SQL Server, Interbase, Informix, or any database system accepting SQL commands. Further, the persistent portion of the data can also be stored as a flat-file.

### 5.4 Physiological Object Analysis and Views

The objects already created may contain, as do breath objects, certain status information determined object-by-object as each object is created. Although this preliminary analysis may be suitable for certain purpose, according to preferred embodiments of this invention, breath objects are further analyzed by examining single breaths in the context of adjacent breaths. This contextual breath-object analysis is advantageous, because, for example, it may provide more accurate analyses of individual breaths, because many types of breath behavior require a more global analysis, and so forth. Examples are latter behaviors are sleep hypopnea and Cheyne-Stokes Respiration (“CSR”). Existing breath analysis systems generally lose much information by considering individual breaths in isolation.

Contextual breath analysis may begin after breath objects, organized in breath-object containers, have been recognized from an input signal and stored in object database 12 (FIG. 1). The further analysis preferably creates further structures, known as “views,” that associate stored breath objects according to predetermined physiological criteria. In FIG. 1, exemplary view are apnea view 13, cardiac view 14, and cough view 15. Preferably, the access object structures representing these views are also made persistent in the database. Optionally, however, these structures may be created when needed to respond to a query and discarded afterwards.

Views and view creation are described next. Again, although much subsequent description focuses largely on pulmonary-related objects, it will be apparent the same analysis structures may be applied to other physiologic modalities.

View Structure

Views, whether persistent or transient, are preferably represented as structured data such as objects, which relate or associate event objects (usually primary event objects) that have been determined to be part of the view. View objects may directly relate all pertinent event objects, or more preferably may indirectly relate event objects through intermediate event group objects. Event group objects are advantageous, for example, in order to represent periods that satisfy the view conditions and include several, usually sequential, events. For example, because a period of coughing may include several coughs, cough view objects would associate cough group objects and each cough group object would further associate those sequential cough events (a cough event being a breath primary object which satisfy criteria for a cough) occurring during the period.

In more detail, Table 7 presents an exemplary class of which event group objects are instances.

TABLE 7
Class: Base Event Group

| Member | Purpose |
|---|---|
| Begin time | Beginning time of this event group |
| End time | Ending time of this event group (event may span |
| | multiple breath objects) |
| Start index | Pointer to first event in this group |
| End index | Pointer to last event in this group |
| Number | Number of events in this group |

Here, the “begin” (“end time”) object data is the time of the BI (EE) event of the first (last) breath object in this event group. Similarly, the “start” and “end index” data are appropriate pointers or addresses to the beginning and ending breath objects in their container object, so that the other objects in the event group are between these objects. “Number” data is the number of breath objects in this group. Optionally, additional information may be derived from the breath objects in the event group and added to the event group object.

View objects in preferred embodiments serve both to construct a requested view and represent the requested view once constructed. Table 8 presents an exemplary class of which view objects are instances.

TABLE 8
Class: Event View Base

| Member | Purpose |
|---|---|
| Event groups | Double-ended queue of event group objects |
| Access breath | Methods for iteration/accessing of breath objects |
| event objects | present in the view |
| Breath container | Breath container holding breath objects |
| ForEach | Virtual method implemented for testing that a given |
| | condition (apnea, etc); actual methods are |
| | implemented in each derived class representing a |
| | particular view |
| Upper/lower | Methods for providing upper and lower quartile range |
| quartile methods | computation |
| Process | Method for scanning breath objects in breath |
| | container, calling ForEach on each breath object |

The first two object members are largely directed to view representation. The “event groups” data associates the event groups with the view object, and the “access breath event objects” method provides for easy access to the breath objects in the view. The remaining object elements are largely directed to view construction. The “breath container” object data associates the breath object container over which the view is to be constructed with the view object. The “foreach” virtual method examines a specific breath object and its neighbors to determine if it is qualified to be in the view being constructed. The “process” method manages searching the associated breath container and applying the foreach method to breath objects in that container (note that the searching need not be done sequentially). Additionally, this class may provide other supporting methods, such as methods for generic inter-quartile computations, method for logging results to HTML pages, methods that support the ForEach method, and so forth. When the foreach method is implemented in a rule-based manner, such supporting functions may include a dynamic rule-set score-board system as known in the art.

Briefly, a scoreboard system includes a scoreboard and rules that can be applied to an event and which return values to a scoreboard. Each event is tested against the rules, and the values returned for an event are added together to generate an overall score for that event, also stored in the scoreboard. If the overall score exceeds a predetermined value, the condition being tested for is assumed to exist for that event.

This view object structure simplifies creation of actual view classes. All that is needed is to provide an appropriate foreach method (overriding the foreach method in the view class) and to create a subclass of the view class that references this foreach method. A particular of particular data view is then an instance of the actual view subclass. Table 9 presents an exemplary subclass of the view subclass of which apnea view objects are instances.

TABLE 9
Subclass: Apnea View (derived from Event View Base)

| Member | Purpose |
|---|---|
| Apnea view | Object constructor performing initialization |
| ForEach | Method testing breath objects for apnea |

Here, the “apnea view” constructor performs apnea-specific view-object initialization, such as for example setting parameters defining apneic breaths for the monitored individual. These parameters might include tidal volume thresholds, time between normal breaths, and so forth, and might differ from individual to individual, for example, with age. The “foreach” method then performs the specific tests that qualify a breath object as apneic.

Similarly, Tables 10 and 11 present representative subclasses for constructing and representing hypopnea and cough views of a breath object container.

TABLE 10
Subclass: Hypopnea View (derived from Event View Base)

| Member | Purpose |
|---|---|
| Hypopnea view | Object constructor performing initialization |
| ForEach | Method testing breath object for hypopnea |

TABLE 11
Subclass: Cough View (derived from Event View Base)

| Member | Purpose |
|---|---|
| Cough view | Object constructor performing initialization |
| ForEach | Method testing breath object for cough |

FIG. 5 illustrates two exemplary view object structures. View structure 80 is a portion of an apnea view constructed over breath container 82, which in turn represents a plurality of breaths 85. This view is represented by apnea view object 81 to which are associated with breath container 82 and event group objects, such as event group 83 and other event groups indicated at 84. Event group 83 associates a contiguous sequence of three apneic breaths, illustrated as the three leftmost breaths of breaths 85. The information representing this association link may be the breath indexes, start index 89 to the beginning breath of this apneic group and end index 90 to the last breath of this group. As already described, each of these apneic breath objects in turn associates, for example, 91, its primitive event objects, and the primitive event objects may point to relevant occurrence times in the signal file data 86. Event group 83 also directly includes beginning 87 and ending times 88 of these apneic breath sequence in the signal file of this apneic breath group.

Next, view structure 95 is a more schematically illustrated, exemplary cough view. Here, the cough view object associates two illustrated event groups, event group N and event group N+1, each of which point to single breath which has been qualified as a cough.

View Creation

To create a new apnea view, for example, an instance of the apnea view subclass is constructed and initialized to point to the container objects over which the view is to be constructed. The process method searches the container applying the apnea foreach method to its breath objects. When a qualified apneic breath object is found, it is added to the current event group if it is a part of a contiguous sequence of apneic breaths. If no such group exists or if this is the beginning of a new apneic breath sequence, a new group object is created, the new group object is added to the apnea view object, and one or more apneic breath objects are then added to the group object.

The foreach methods, which qualify breath objects for inclusion in a view, are implemented according to the particular breath classification and qualification problem posed by the view. For some views, for example apnea views, detailed examination of the characteristics of individual breaths may be sufficient, while for other views, for example Cheyne-Stokes respiration views, examination of the pattern of several adjacent breaths may be needed. In many cases, either approach may be implemented in different embodiments.

Briefly, in certain embodiments, foreach methods may recognize and classify apneas by detailed examination of the properties of individual breaths, an apnea being recognized if the duration of the breath from the initiating BI primitive event to the terminating EE primitive event is sufficiently long and if the tidal volume is sufficiently small when compared with a concurrent baseline. Information needed for this examination may be stored as elements/members in the individual breath objects (see Table 3). A breath object recognized as apneic may be further classified as central or obstructive by examining the RC and AB signal data accessible through the breath object. Generally, if the RC and AB signals have approximately normal amplitude but are out of phase, the apnea is considered obstructive, while if both these signals have significantly decreased amplitude, the apnea is considered central. Mixed patterns of RC and AB signals may be considered to reflect mixed apneas. Hypopneas may be recognized and classified as breath objects with amplitudes and durations intermediate between normal baseline values and the apneic threshold values. See, for example, U.S. Pat. No. 6,015,388, issued Jan. 18, 2000 (methods for determining neuromuscular implications of breathing patterns); and U.S. Pat. No. 4,777,962, issued Oct. 18, 1988 (methods for distinguishing types of apneas by means of inductive-plethysmogrpahic measurements).

Such single breath apnea and hypopnea recognition may be supplemented or confirmed (or replaced) by examining the patterns of several sequential breaths. Patterns may be conveniently expressed in a regular-expression like notation that specifies sequences of breath objects with sequences of particular properties; and sequences of breath objects instantiating a pattern may be recognized in breath-object containers by use of finite state machines. For example, recognition of an apneic or hypopneic breath object may be confirmed by finding a pattern of normal breath objects, or even breath objects with increased amplitude, surrounding the recognized apneic or hypopneic breath object.

Certain types of respiratory events may be best recognized as patterns of breath objects instead of by examination of individual breath objects. For example, some cough may be defined by a pattern of a few unusually short breath objects among otherwise normal breath objects. Alternatively, a cough may be recognized by analysis of individual breath objects searching, for example, for breath objects with unusually high air flow. Finally, further types of respiratory events can only be seen in breath patterns. For example, Cheyne-Stokes respiration (“CSR”), which is defined as a breathing pattern characterized by rhythmic waxing and waning of respiration depth over several sequential breaths perhaps with regularly recurring apneic periods, can only be recognized by seeking appropriate patterns of several breath objects. CSR cannot be recognized from single breath objects alone.

Finally, in alternative embodiments, foreach methods may use known rule-based methods to combine the advantages of single-breath examination with breath pattern searching. For example, for hypopnea qualification, certain rules may have predicates (if clauses) that depend on parameters of an individual breath object being tested, and consequents (then clauses) that provide a likelihood score that the tested breath object represents a hypopneic breath. Other rules may have predicates including patterns that are matched against breath objects that are in the vicinity of the tested breath object, and consequents providing further likelihood scores in case the patterns do or do not match. The likelihood scores may be accumulated in a score-board data structure, and linear or non-linear combinations of the scores tested against thresholds to finally qualify the tested breath as hypopneic or not. Further, it may be advantageous for various views to be constructed together in order that rules for various breath types may be evaluated and their scores tested together. Other rule based methods known in the art may also be employed.

### 5.5 Further Embodiments and Options

The methods, data, and object structures have been described in detail above in an implementation primarily for representing and processing pulmonary information. However, these methods and structures are flexible and modular, and one of skill in the art will understand how they may be readily adapted to the representation and analysis of other physiologic processes that can be characterized by time varying parameters including, for example, capnometery (measurements of carbon dioxide concentration), electroencephalography (EEG), electrooculography (EOG, measurements of ocular muscle activity), electromyography (EMG, measurements of muscle activity), sound microphone(s) measurements (for detecting sounds associated with cough, snoring, etc), body temperature, accelerometers (preferably positioned on the legs or torso and for determining position and activity), blood glucose concentration, blood pressure, blood flow, etc. Physiological aspects of these processes are defined by primitive and primary physiological events as known in the art, and data structures may be created to represent such primitive and primary events as described for respiratory events.

Particularly illustrative of this flexible applicability are applications to cardiac and pulse oximetry data, both of which are now briefly described. Cardiac data is much like pulmonary data, being characterized by volume information, such as stroke volumes, derivable from ambulatory thoracocardiographic (TCG) data, and by timing information, such as RR interval times, derivable from electrocardiographic (ECG) data. Pulse oximetry data may be characterized by arterial oxygen saturations and desaturations measurable in, for example, a finger.

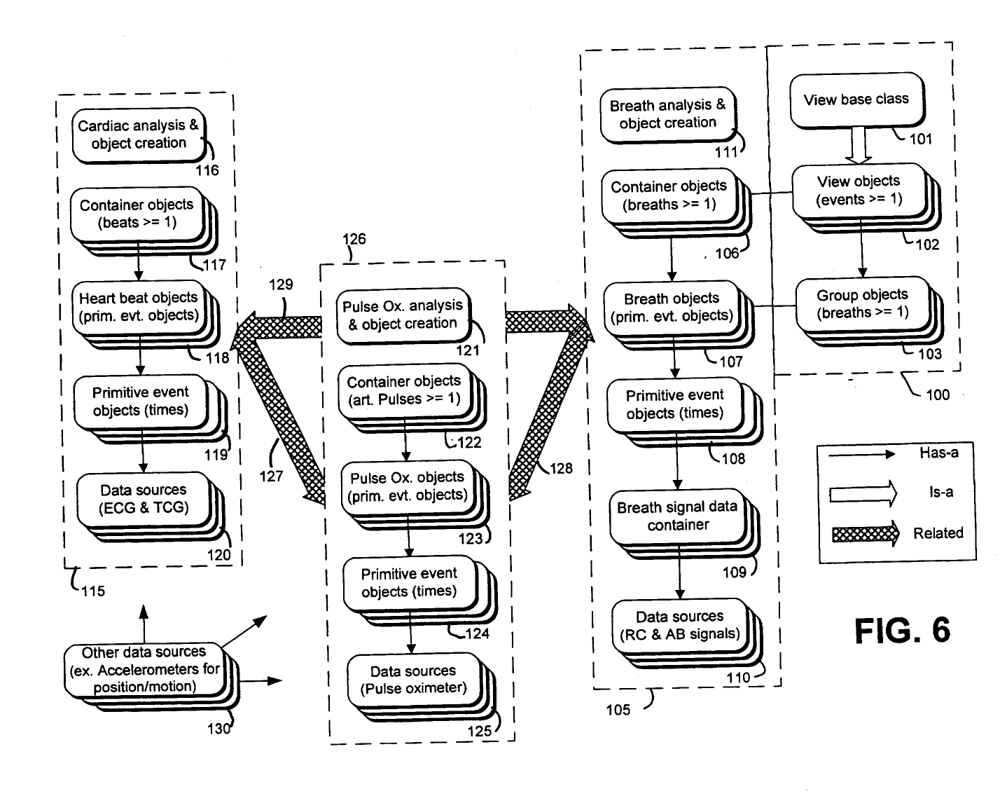

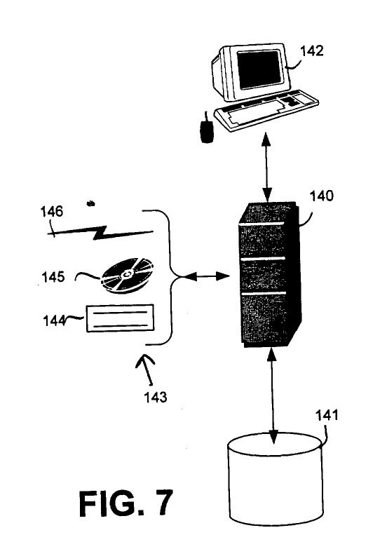

FIG. 6 schematically and summarily illustrates object structures for cardiac and pulse oximetry data along with details of the already described pulmonary object structures. Here, cardiac 115 and pulse oximetry 126 object structures are implemented similarly to their corresponding pulmonary structures. Because all three types of signals have similar general characteristics, all these implementations include a hierarchy of object instances generalizing aspects of their periodic input signals. These objects are instances of corresponding classes, and the objects and classes may be structured by inheritance of common characteristics. However, each hierarchy has data and methods that are specific to the processes being represented.

Turning first to pulse oximetry data, methods and data for recognizing pulse oximetry signals and creating pulse oximetry objects are preferably structured as instances of analysis and object creation class 121. These instances would include methods for filtering input pulse oximetry signals, for recognizing primitive signal events, and for grouping such primitive events into arterial pulse oxygen saturation events. Representative object structures are preferably created during this processing.

At the top of the object hierarchy are, as with the pulmonary representation, container objects 122 which group data from many pulses that are related by being, for example, part of a single measurement session, or by occurring during a period of homogenous patient activity or posture, or the like. Next in the hierarchy are objects 123 which represent the actually observed arterial pulses and their oxygen saturation, and which are instances of the class presented in Table 12.

TABLE 12
CLASS: ARTERIAL PULSE

| Member | Purpose |
|---|---|
| Associated primitive | Associated objects describing pulse oximeter |
| arterial pulse | signal characteristics or phases; may be stored |
| events/phases | as separate, associated objects, or may be |
| | stored in this object |
| Time | Methods/data returning the time of a |
| | particular arterial pulse |
| O₂ saturation | Methods/data returning the observed O₂ |
| | saturation level (preferably as a percent of a |
| | maximum saturation); may also provide a |
| | running saturation baseline |
| O₂ desaturation | Methods/data returning the desaturation level |
| | of this pulse below baseline and its duration |
| Body posture data | Methods/data returning an indication of the |
| (optional) | subject's posture and activity at the time of |
| | this pulse (for example, a code indicating: for |
| | position, recumbent on the left or right side, |
| | or on back or front; and for activity, sitting, |
| | standing, sleep, awake, walking, running; and |
| | the like) |
| Good or artifactual | Status flags including indicia of, for example, |
| | whether this pulse has good saturation data, or |
| | is an artifact. |
| Breath event data | Dynamic arrays of pointers to breath event |
| | objects (usually only one) associated with this |
| | arterial pulse |
| Heart event data | Dynamic arrays of pointers to heart event |
| | objects (usually only one) associated with this |
| | arterial pulse |

Briefly, in one preferred embodiment, each observed arterial pulse is formed from a group of its associated primitive pulse events 124. These primitive events may represent portions of a pulse oximeter signal that correspond to, for example, the beginning of a pulse, its up stroke, its peak magnitude, its down stroke, and its termination, and that may include, for example, the event time and characteristics such as magnitudes or rates. Arterial pulse objects 125 are then created and initialized when a pattern matching engine, perhaps based on state machines or other periodic signal recognition techniques, recognizes a sequence of primitive events defining an arterial pulse. In another embodiment, only pulse objects are persistently stored; primitive event objects, if created, as discarded. Preferably, the arterial pulse is associated with a concurrently measured blood pressure, which may also be stored as part of the arterial pulse object

Oxygen saturation methods and data represent the arterial oxygen saturation observed for the current pulse, and may preferably include a present value of a running saturation baseline. Such a baseline may, for example, be the median of saturation values in a 2-4 min. window including the current pulse, so that deviations from this baseline can be recorded in the pulse object. Of particular interest are negative deviations (desaturation) of the current saturation from the running baseline, and desaturation information including its magnitude and duration may be stored in the pulse object. Because oxygen saturation/desaturation can be affected by body posture and activity, posture and activity indications are also preferably stored in arterial pulse objects (and optionally also in breath and heart beat objects). Posture and activity data can be obtained from concurrent recordings of one or more accelerometers attached to the subject. Also, pulse objects may include flags (or other indicia) indicating whether or not this pulse object represents good data or artifact, as determined, for example, by checking that the associated primitive events have acceptable timing and magnitudes.

Additionally, arterial pulse objects preferably include data identifying concurrently occurring (or otherwise related) breath objects and heartbeat objects. These latter objects may also include data identifying related objects of the other types. Generally, these relationships may be many-to-many, and are generically so illustrated in FIG. 6 as double-headed, cross-hatched arrow 128 relating pulses to breaths and as arrow 127 relating pulses to heart beats. Typically, each breath is usually associated with many pulses, so that relationship 128 is at least one-breath-to-many, but may be many-to-many since these processes are not in temporal synchronism. Again typically, each pulse is usually associated with one heart beat, so that relationship 127 is one-heart-beat-to-one-pulse, but again because of arrhythmias and other problems this synchronism may be lost. Also illustrated by arrow 129 is a relationship between breath objects and heart beat objects, which, although usually one-breath-to-many-heart-beats, again may be many-to-many because the processes are not in temporal synchronism.

It should be understood that the associations intended herein are preferably defined in physiologic terms, and are not simply limited, for example, to links between specific, well-defined objects. For example, breathing and cardiac activity may be subject to concurrent neural or other physiological control, in which case an association between breaths and heartbeats would be defined by their occurring at overlapping times. Also, breathing difficulties may lead to anxiety having widespread physiological consequences, and here heartbeats (and also, for example, EEG activity) would lag their associated breaths by perhaps 5 secs to 1 min. or more (time for perception and response to anxiety). In pulse oximeter data, an arterial pulse typically lags its causative heartbeat by a known time delay (blood transit time from the heart to the artery) so that the heartbeat associated with an arterial pulse would precede the pulse by this time delay. Further, apneas or other breath disturbances may lead to oxygen desaturation in arterial pulses beginning perhaps 5 to 10 secs later (blood transit time from the lungs to the measured artery), thus leading to a still another type of association. Moreover, physiological association may be to a greater or lesser degree “fuzzy”. In the case of breathing abnormalities and consequent arterial desaturation, a range of a few abnormal breaths may be related to a range or a larger number of arterial pulses. On the other hand, it is usually possible to associate a single arterial pulse object with its causative heartbeat object.

Further, association and relations between objects may be manually created by a user who views the various data types.

Association implementations are preferably chosen in view of these physiological facts. More specific, less fuzzy, associations may be defined by single pointers, or by small groups of pointers, between single objects or between temporally a few contiguous objects. More fuzzy associations may be implemented as pointers between groups of related objects. Alternatively, associations may be implemented using occurrence times: objects of one type occurring in a certain range of time may be related to objects of another type occurring in another range of time, where the time ranges are appropriate to the physiological association being implemented.

Lastly, data sources 125 encapsulate the actual pulse oximeter data inputs, and may include as for pulmonary objects, data storage containers providing for access to raw input data.

Cardiac methods and objects will be briefly described, because they are preferably structured similarly to the already-described breath and pulse oximeter objects. Cardiac signal recognition and object creation methods may be grouped as instances of object creation class 116. Container objects 117 serve to group heart beat objects that are related by, for example, being observed during a single recording session or present in a single recording data file. The heart beat objects 118 include methods and data returning the characteristics of observed heart beats, and preferably also include (or include information 127 and 129 that relates them to) their component primitive cardiac event objects, and to concurrent or otherwise related breath 107 and arterial pulse objects 123.

Data source structures or objects encapsulate access to raw cardiac data sources, and may include provisions for real time data access as well as later access to specified portions of the raw data (as do breath signal container objects 109). Cardiac data is processed by a heart detection engine, which, in a simple embodiment, may analyze a two-lead ECG signal to find R-wave peaks, measure R-R intervals, and may create heart beat objects 118 including heart beat interval data along with a running baseline heart rate. In a further embodiment, a heart engine may analyze multi-lead ECG data to create primitive event objects for each portions of an ECG trace, i.e., the P-wave, the QRS-complex, and the T-wave, and then to create heart beat objects with information about the character of the ECG pattern. Additionally, TCG signals from a mid-thoracic inductive plethysmographic band may be processed to provide indicia of cardiac output and ventricular wall motion, and these indicia integrated in heart contraction objects along with characteristics of the ECG pattern.

In more detail, in different embodiments, different primitive (and primary) cardiac events recognized. For example, if ECG signals are an available cardiac data source, the recognized primitive events may simply be the electrical depolarization and repolarization phases of a cardiac cycle. The depolarization phase may be further recognized as having an atrial depolarization phase—e.g., the P wave phase—and a ventricular depolarization phase—e.g., the QRS complex phase. Furthermore, the primitive ventricular depolarization phase may be recognized by resolving the QRS complex into its component phases—e.g., the Q wave phase, the R wave phase, and the S wave phase. Alternatively, the QRS complex phase may be further recognized and described by use of vector-cardiographic data and techniques. Then the repolarization phase may be further recognized as having a ventricular repolarization phase—e.g., the T wave phase. If TCG signals are also available, the recognized primitive cardiac events may also include a diastolic phase followed by a systolic phase. These phases correspond to physical cardiac pulsations, and may be related to the concurrent electrical phases available from the ECG signals.

Usually, cardiac primary events are individual heart beats, or complete cardiac cycles, although other primary cardiac events may be built from recognized primitive events. Heart beat objects preferably include elements summarizing their electrical characteristics, for example, their conduction times and patterns, and their functional characteristics, for example, indicia of stroke volume and wall motion (that may be derived from TCG data).

Finally, views may be defined for selecting patterns of heartbeats with selected properties. Common cardiac views may represent instances of normal or abnormal cardiac rhythms. Views may be defined for abnormal rhythms from ectopic beats and premature ventricular contractions, to conduction defects, to atrial or ventricular arrhythmias, and the like. Views may also be defined for periods of ECG abnormalities, such as periods of ST segment elevation. Views may also be defined to select functional cardiac characteristics, such as periods of unusually low or high cardiac output, periods of abnormal or paradoxical wall motion, and the like.

Although not illustrated, additional view classes are preferably created which may be instantiated to provide access to various aspects of the additional physiologic data types. For example, a cardiac view may examine heart beat objects for patterns of variability in cardiac output or in heart rate, and return objects providing direct user access to periods of such variability. Specifically, such views may provide indicia of episodic arrhythmias (for example, atrial fibrillation, premature ventricular contractions, respiratory sinus arrhythmia, and the like), transient ischemia, and so forth. Pulse oximeter views may, for example, return objects accessing arterial pulse objects having oxygen desaturations below a specified amount below the running baseline, and so forth.

Importantly, the present invention provides the novel ability to view data representing multiple concurrent physiological processes (whether or not in object-oriented structures) in a monitored subject. Such views could be used to search for physiological correlations by selecting objects from one process according to specified characteristics and then accessing temporally related objects from other process. Thereby perturbations in the other processes that are associated with the certain characteristics of the first process may be examined. Such views could also be used to find occurrences of known correlations by selecting related objects from two processes that have correlated characteristics. Efficient construction of views across multiple physiological processes is facilitated by explicit relationships (temporal or otherwise) between physiologic objects of different types illustrated by arrows 127, 128, and 129 in FIG. 6.

In particular, it has been found that views combining breath objects with related non-respiratory event objects, such as heart beat objects and pulse objects, can provide significant and useful new information. For example, views combining arterial pulse objects with breath and cardiac objects can determine relationships between periods of arterial desaturation events and characteristics of concurrent cardiac and pulmonary processes. Thereby, the severity of arterial desaturation and any consequent changes in cardiac activity may be linked to characteristics of periods of apnea of hypopnea apparent in breath object views.

For another example, coughs may often produce breath patterns that are quite similar to yawns and sighs. However, it has been found that real coughs often result in an transient and sudden drop (up to approximately 20 beats per min) in heart rate at the end of expiration terminating a cough. Accordingly, a cough view may use rate data in heart beat objects 118 associated 129 with a breath object 107 as a further determining factor in cough detection or verification. Additionally, true oxygen saturation signals may be separated from artifact by correlation with heart beat information. Accordingly, pulse object creation may be made more reliable by correlation 127 with heart beat information. See, for example, U.S. Pat. No. 5,588,425, issued Dec. 31, 1996 (methods for improved interpretation of pulse oximetry processing).

In additional embodiments, additional types of physiological processes may be represented by the methods and structures of this invention. For example, when a person with COPD has a coughing attack, the blood may desaturate to dangerous levels and the heart rate may increase. This changes can lead to anxiety which may worsen the sense of breathlessness and the physiological response. Accordingly, for such subjects, recording and representation of electroencephalographic wave patterns (EEG) may be advantageous in assessing their levels of anxiety. Such representation may include time changes in the power in the standard EEG frequency bands, such as the alpha band, which can provide indicia of anxiety and stress.

### 5.6 Systems and Databases

In addition to the methods described above, this invention also includes programs for configuring computer systems to perform these methods, computer systems configured for performing these methods, and computer-readable memories, both transient and persistent, configured with the object structures of this invention.

FIG. 7 schematically illustrates an exemplary system for performing the present invention. One of skill in the art will understand that there are wide range of specific system structures that are functionally equivalent to the illustrated system that may also perform the present invention. The exemplary computer system includes one or more server computers 140 connected to one or more permanent computer-readable memories, such as disks 141, to user interface equipments 142, and to various external communication devices 143. The server computers 140 routinely include transient computer-readable memories, such as RAM, for holding programs and data. External communication may proceed equivalently by means of telecommunications, such as the Internet 146 (including wireless links), or of removable computer-readable media, such as CD-RW/ROM 145, or of memory cards 144. These communication devices may receive the various types of physiologic data processed by this invention's methods and may also exchange program products and structured databases. These systems may be managed by standard operating systems, such as Linux or Windows. Databases on computer-readable media may be managed by standard database management systems such as commercial RDBMS including Oracle 9i, Microsoft SQL Server, Interbase, Informix, or any database system accepting SQL commands. Further, the persistent portion of the data can also be stored as a flat-file.

Programs for configuring computers to perform this invention's methods may be written in a convenient object-oriented language, such as Java or C++, compiled, and loaded into the (RAM) memory of computers 140 for execution. In non object-oriented embodiments, the C language may be preferred. These programs may be exchanged, for example as program products, by various communication devices, such as devices 143.

Execution of the methods of this invention result in configurations of structured objects stored in computer-readable memories, both transient (such as RAM contained in computer 140) and persistent (such as media 141, 144, and 145). This invention includes such structured computer memories. Preferred memory structures are presented in the following Tables 13, 14, and 15, and their relationships are illustrated in FIG. 6. These memories may includes all or only part of the structures to be described; this invention is not limited to memories configured with all the described structures in the preferred combination.

With reference to FIG. 6, Table 13 presents preferred breath-related objects (and classes) 105 and breath-related view objects (and classes) 100, and summarizes their contents.

TABLE 13
Breath Objects

| Object | Description |
|---|---|
| Breath container 106 | A searchable container object in which are stored breath objects |
| Breath object 107 | An object representing a single breath including information |
| | about the quality and type of breath, times of the next breath |
| | and the next good breath, and so forth |
| | Optionally may be associated with heart beat objects 118 |
| | representing concurrent heart beats and pulse objects 116 |
| | representing concurrent arterial pulses |
| Breath primitive | An object representing a single primitive breath event (such |
| event object 108 | as beginning of inspiration, peak inspiratory flow, and the |
| | like) in an input signal and including lung volume signal |
| | parameters and time |
| Breath data | Containers for input signals and connections to signal |
| container 109 & | sources (may or may not be structured as objects) |
| data sources 110 | |
| Breath analysis & | An object including methods for input signal analysis and |
| object creation 111 | breath object creation |
| View base class 101 | A base class for all breath-object views defining |
| | associations to events found to be in the view and methods |
| | for searching or iterating the objects in the view |
| | Optionally, may define links to events having related heart |
| | beat objects 118 and pulse objects 116 |
| View object 102 | An instance of a subclass of the view base class that |
| | provides data and methods to recognize events in particular |
| | views; for example, sleep hypopnea and sleep apnea views |
| | Optionally the view methods may examine heart and pulse |
| | objects, 118 and 116, as well as breath objects |
| Event group object | An object container for a contiguous group of breath objects |
| 103 | 107 found to be in a view |

Next, Table 14 presents preferred cardiac-related or heart-beat-related objects (and classes) 115, and summarizes their contents.

TABLE 14
Heart Beat Objects

| Object | Description |
|---|---|
| Heart beat object | A searchable container object in which are stored all heart |
| container 117 | beat objects found in an input signal |
| Heart beat object 118 | An object representing a single heart beat including |
| | information about the quality and type of ECG waves, and |
| | optionally TCG information about cardiac output and ventricular |
| | motion |
| | Optionally may be associated with breath objects 107 |
| | representing concurrent breaths and pulse objects 123 |
| | representing concurrent arterial pulses |
| Heart beat | An object representing a single heart beat primitive event |
| primitive event | (such as start of P-wave, peak of R-wave, start of ventricular |
| object 119 | contraction, peak flow, and the like) in an input signal and |
| | including cardiac input signal parameters and time; |
| | optionally also primitive TCG signal events |
| Heart beat data | Encapsulates access to input cardiac signals and provides |
| sources 120 | later access to raw cardiac data (may or may not be |
| | structured as objects) |
| Heart beat analysis | An object including methods for input signal analysis and |
| & object creation 119 | heart beat object creation; for example including methods |
| | for ECG and TCG processing |
| Heart beat view | A base object for all heart beat object views defining |
| base class | associations to events found to be in the view and methods |
| (not illustrated) | for searching or iterating the objects in the view |
| | Optionally, may define links to events having related breath |
| | objects 107 and pulse objects 123 |
| Heart beat view | An instance of a subclass of the view base class that |
| object | provides data and methods to recognize events in particular |
| (not illustrated) | views |
| | Optionally the view methods may examine breath and pulse |
| | objects, 107 and 123, as well as breath objects. |
| Heart beat event | An object container for a contiguous group of heart beat |
| group object | objects 118 found to be in a view. |
| (not illustrated) | |

Finally, Table 15 presents preferred pulse-related or pulse-oximeter-related objects (and classes) 126, and summarizes their contents.

TABLE 15
Pulse Oximeter (Arterial Pulse) Objects

| Object | Description |
|---|---|
| Pulse-oximeter | A searchable container object in which are stored all Pulse- |
| object container 122 | oximeter objects found in an input signal |
| Pulse-oximeter | An object representing a single arterial pulse; described with |
| object 123 | respect to Table 12 |
| | Optionally may be associated with breath objects 107 representing |
| | concurrent breaths and heart-beat objects 118 |
| | representing concurrent cardiac contractions |
| Pulse-oximeter | An object representing a single pulse-oximeter primitive |
| primitive event | event; described with respect to Table 12 |
| object 124 | |
| Pulse-oximeter | Encapsulates access to input pulse-oximeter signals and |
| data sources 125 | provides later access to raw cardiac data (may or may not be |
| | structured as objects) |
| Pulse-oximeter | An object including methods for input signal analysis and |
| analysis & object | arterial pulse object creation by processing pulse oximeter |
| creation 121 | signals |
| Pulse-oximeter | A base object for all pulse-oximeter object views defining |
| view base class | associations to events found to be in the view and methods |
| (not illustrated) | for searching or iterating the objects in the view |
| | Optionally, may define links to events having related breath |
| | objects 107 and heart-beat objects 118 |
| Pulse-oximeter | An instance of a subclass of the view base class that |
| view object | provides data and methods to recognize events in particular |
| (not illustrated) | views |
| | Optionally the view methods may examine breath and |
| | cardiac contraction objects, 107 and 118, as well as breath |
| | objects. |
| Pulse-oximeter | An object container for a contiguous group of pulse- |
| event group object | oximeter objects 123 found to be in a view. |
| (not illustrated) | |

Preferred relationships among these objects are illustrated in FIG. 6, where a line arrow illustrates a association of objects, a hollow arrow illustrates a class-sub-class relationship, and a hollow cross-hatched arrow illustrates relationships between objects of different modalities. Considering first breath objects 105, the association of container objects 106 with breath objects 107 and of breath objects 107 with primitive event objects 108 is typically one-to-many, but in particular cases, one or all of these may instead be one-one. Thus a database memory includes one or more breath container objects 106. Each breath container object usually associates a plurality of breath objects 107 in the memory. In turn each breath objects associates the primitive event objects in the memory and representing signal events from which the breath represented by the breath object is composed. Each primitive event object includes event times, which may be used to find the associated portions of the input signal data in signal data containers 109. The data container may have a non-object structure; for example, it may be a file.

The data container 109 and remaining breath objects and structures will typically be in a database memory where input signals are being recognized and new breath objects are being created, but may not be present in a database memory used solely for object analysis. Data containers 109 preferably present a sufficiently generic interface for data retrieval so that the data and methods of the primitive event objects are reasonably independent of the details of actual data sources. The relationship between data containers 109 and data sources 110 may not object-structured, being directly made by conventional procedure invocations, sub-routine calls, and the like. Lastly, the breath analysis & object creation object 111 serves primarily as a structure used to create the other breath objects, and need not be present in an already-created database used solely for analysis purposes. In certain embodiments, this object may be sub-class of a general class defining cross-modality creation of physiologic objects.

Next, breath view objects 100 includes view base class 101 and the view objects 102 representing particular views. The base class gathers general data and methods (including virtual methods) for creating and representing views, while its sub-classes have specific methods and data for creating particular views that answer particular user queries. Next, view objects 102 are preferably instances of these sub-classes and serve to create and represent particular views. Since views are generally represent queries concerning objects in containers, view objects 102 are preferably associated with the one or more breath container objects 106 over which they are built. View objects also provide access to those breath objects qualified to be part of the view by means of one or more associated event group objects 103, which associate one or more temporally sequential breath objects 107 that are part of a view. In summary, a database typically includes the breath view base class along with subclasses for creating particular views of interest. Additional subclasses can be added to time-to-time to answer various user queries. The subclasses generally have one or more view object instances, each representing a view into one or more breath containers. View objects provide access to breath objects in a container by one or more associated group objects which are in turn associated with one or more breath objects.

FIG. 6 also illustrates more briefly objects and classes (excluding view objects and classes) representing other repetitive physiologic activities, including heart beats 115 and arterial pulses 126. Additional physiologic processes that can be represented in the databases include those measured by, for example, capnometery, EEG, EOG, EMG, sound microphone(s), body temperature, accelerometers, and blood glucose concentration.

Generally, objects representing each separate physiological modality are structured similarly to the objects representing breaths and have similar associations. Importantly, where two or more physiological modalities are present in a database, associations between events of different modalities may be constructed and provide additional useful information. FIG. 6 illustrates exemplary cross-modality associations of temporally concurrent objects. For example, each heart beat object 118 will typically be associated 127 with one arterial pulse object 123, and conversely. However, since each breath typically spans several heart beats, association 129 between breath objects 107 and heart beat objects 118 is usually one-many. Conversely, since the occasional heart beat may span two sequential breaths, each heart beat object may be associated with up to two breath objects. Finally, breath objects may be associated with pulse objects 123 similarly to their association with heart beat objects. Therefore, cross-modality associations, such as associations 127, 128, and 129, may be one-to-one, one-to-many, or many-to-many in different cases.

In alternative embodiments, associations of types and complexity different from those illustrated in FIG. 6 may be advantageous. In particular, alternative embodiments may make more extensive use of class-sub-class relationships. Also, in certain embodiments it may not be necessary to create and store primitive events objects, the data normally present therein being immediately used in the creation of the primary event objects. Further, in certain embodiments, container objects may be omitted. Here, the grouping of primary event objects would be by other means, for example, by being in separate database files, by being sequentially linked, or so forth. Finally, this invention may be implemented without explicit object structures. In such embodiments, the described modularity and data relationships would be simulated by pointers, indexes, and the like as has been long well known in the art. Explicit object structures can primarily serve to automate and enforce structures that could be created and maintained with prior programming techniques.

For certain elementary types of physiological data, a direct representation may be advantageous. For such data, separate representation objects would not be constructed; instead this data would simply be stored as part of the characteristics of the primary event objects that are created. Accordingly, FIG. 6 illustrates such other data sources 130 as directly providing input to the creations of the other primary event objects without separate representation. An example of such data is accelerometer data defining position and activity. Accelerometer data may be directly processed to provided indicia of position and activity which are then stored directly in the cardiac, breath, and arterial pulse objects. Table 12 illustrates pulse oximeter objects with accelerometer-derived data.

Such other alternatives for storing and representing physiological processes in a modular and hierarchical within the scope and spirit of the above-described invention as will be apparent to one of average skill in the art and within the scope of the appended claims.

The invention described and claimed herein is not to be limited in scope by the preferred embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference for all purposes. Further, none of these references, regardless of how characterized above, is admitted as prior to the invention of the subject matter claimed herein.

What is claimed is:

1. A computer-readable memory including structured data representing physiological events reflected in input signals from a monitored subject, wherein the structured data comprises:
at least one instance of a data-structure representing characteristics of an elementary-type physiological event, wherein an elementary-type physiological event is defined in dependence on a portion of the input signal, and
at least one instance of a data-structure representing characteristics of a primary-type physiological event, wherein the primary-type event is defined in dependence on one or more elementary-type events.

2. The memory of claim 1 wherein primary-type events are defined in dependence on one or more temporally-contiguous elementary-type events.

3. The memory of claim 1 wherein an elementary-type data-structure instance representing an elementary-type physiological event comprises identification of the portion of the input-signal defining the represented elementary-type event.

4. The memory of claim 1 wherein a primary-type data-structure representing a primary-type physiological event comprises identification of the elementary-type data-structures instances representing the defining elementary-type events.

5. The memory of claim 1 further comprising an instance of a software object that comprises an elementary-type or a primary-type data-structure instance.

6. The memory of claim 1 wherein a primary-type data-structure instance comprises an elementary-type data-structure instance.

7. The memory of claim 1 further comprising at least one instance of a view-type data-structure representing at least one elementary-type or primary-type data-structure instance, the view-type data-structure instance representing one or more physiological events that satisfy a pre-determined physiological criterion.

8. The memory of claim 7 further comprising an instance of a software object that comprises a view-type data-structure instance, the software object being an instance of an object class also comprising a method for accessing the elementary-type or primary-type data-structure instances represented by the view-type data-structure instance.

9. The memory of claim 7 wherein a view-type data-structure represents at least one instance of an event-group data-structure, the event-group data-structure representing one or more temporally contiguous primary-type data-structure instances satisfying the pre-determined physiological criterion.

10. The memory of claim 7 wherein the input signal reflects pulmonary events, and wherein the pre-determined physiological criterion define one or more of a period of apnea, a period of hypopnea, a cough, or a period of Cheyne-Stokes respiration.

11. The memory of claim 1 wherein the input signal reflects pulmonary events, wherein the elementary-type physiological events are breath phases, and wherein the primary-type physiological event are breaths defined by a sequence of breath phases.

12. The memory of claim 11 the breath phases comprise one or more of a begin inspiration phase, or a begin inspiratory flow phase, or a peak inspiratory flow phase, or a peak phase, or a begin expiratory flow phase, or a peak expiratory flow phase, or an end expiration phase.

13. The memory of claim 1 wherein the input signal reflects pulse-oximeter measurement events, and wherein the primary-type physiological events are pulses.

14. The memory of claim 1 wherein the input signal reflects cardiac events, and wherein the elementary-type physiological events are cardiac phases, and wherein the primary-type physiological event are heartbeats defined by a sequence of cardiac phases.

15. The memory of claim 14 wherein the cardiac phases comprise depolarization phases, or repolarization phases, or ECG P-wave phases, or ECG QRS-complex phases, or ECG T-wave phases, or diastole phases, or systole phases.

16. The memory of claim 14 wherein the primary-type data-structures comprise indicia of ventricular volume.

17. The memory of claim 1 part of all of which is included in a RAM storage medium of a computer system or a permanent storage medium of a computer system.

18. The memory of claim 1 part or all of which is included in a removable storage medium that can be transported between computer systems.

19. The memory of claim 1 further comprising data representing the input signals.

20. The memory of claim 1 wherein the physiological events are generated by a respiratory process, or a cardiac process, or a pulse oximetry process, or a capnometery process, or an electroencephalographic process, or an electrooculographic process, or a electromyographic process, or a sound microphone signal process, or a body temperature signal process, or an accelerometer signal process, or a blood glucose concentration signal process.

21. A computer-readable memory including structured data representing physiological events generated by at least a first and a second physiological process, the physiological events being reflected in input signals from a monitored subject, wherein the structured data comprises:
at least one instance of a data-structure representing characteristics of a primary-type physiological event generated by the first process that is defined in dependence on one or more corresponding elementary-type physiological events, wherein the elementary-type events are defined in dependence on portions of the input signal,
at least one instance of a data-structure representing characteristics of a primary-type physiological event generated by the second process that is defined in dependence on one or more elementary-type physiological events, wherein the elementary-type events are defined in dependence on portions of the input signal,
wherein at least one primary-type data-structure instance comprises data identifying at least one other primary-type data-structure instance, the primary-type data-structure instances representing primary-type events of the first and second processes that are physiologically associated.

22. The memory of claim 21 wherein the first and second physiological processes are a respiratory process, or a cardiac process, or a pulse oximetry process, or a capnometery process, or an electroencephalographic process, or an electrooculographic process, or a electromyographic process, or a sound microphone signal process, or a body temperature signal process, or an accelerometer signal process, or a blood glucose concentration signal process, or a blood pressure process, or a blood flow process.

23. The memory of claim 21 further comprising at least one instance of a view-type data-structure representing
(i) one or more primary-type data-structure instances representing events generated by one of the physiological processes that satisfy a pre-determined physiological criterion, and
(ii) one or more primary-type data-structure instances representing events generated by the other of the physiological processes that are physiologically associated with the represented primary-type data-structures.

24. The memory of claim 21 further comprising at least one instance of a first-process primary-type data-structure that comprises identification of at least one further data-structure instance representing characteristics of a first-process elementary-type physiological event.

25. The memory of claim 24 further comprising at least one instance of a second-process primary-type data-structure that comprises identification of at least one further data-structure instance representing characteristics of a second-process elementary-type physiological event.

26. The memory of claim 21 wherein the data-structure instances of the first and second physiological processes are physiologically associated if the represented events have overlapping occurrence times.

27. The memory of claim 26 wherein the primary-type data-structures represent heartbeats and breaths.

28. The memory of claim 26 wherein the primary-type data-structures represent respiratory events, or cardiac events, or arterial pulse events, or capnometery process events, or electroencephalographic process events, or electrooculographic process events, or electromyographic process events, or sound microphone signal process events, or body temperature events, or accelerometer signal events, or blood glucose concentration events, or blood pressure events, or blood flow events.

29. The memory of claim 21 wherein the data-structure instances of the first and second physiological processes are associated if the represented events have occurrence times displaced by a determined amount.

30. The memory of claim 29 wherein the primary-type data-structures represent heartbeats and arterial pulses, and wherein the determined time displacement is a transit time of blood from the heart to the measured artery.

31. The memory of claim 29 wherein the primary-type data-structures represent breaths and arterial pulses, and wherein the determined time displacement is a transit time of blood from the lungs to the measured artery.

32. The memory of claim 29 wherein the primary-type data-structures represent heartbeats and breaths, and wherein the determined time displacement is an anxiety reaction time.

33. The memory of claim 21 wherein the data-structure instances of the first and second physiological processes are physiologically associated if the represented events have a cause-and-effect relationship.

34. The memory of claim 21 wherein the data-structure instances of the first and second physiological processes are manually associated by a user.

35. A computer-implemented method for analyzing input signals from a monitored subject, the input signals reflecting physiological events in the subject, the method comprising:
recognizing at least one elementary-type physiological event in dependence on a portion of the input signal,
creating at least one instance of an elementary-type data-structure representing characteristics of the elementary-type event,
recognizing at least one primary-type of physiological event from one or more contiguous elementary-type events, and
creating at least one instance of a primary-type data-structure representing characteristics of a primary-type event.

36. The computer-implemented method of claim 35 wherein the physiological process is a respiratory process, or a cardiac process, or a pulse oximetry process, or a capnometery process, or an electroencephalographic process, or an electrooculographic process, or a electromyographic process, or a sound microphone signal process, or a body temperature signal process, or an accelerometer signal process, or a blood glucose concentration signal process or a blood pressure process, or a blood flow process.

37. The computer-implemented method of claim 35 wherein the input-signal characteristics comprise a maximum value of a portion of the signal, or a minimum value of a portion of the signal, or a maximum value of the time derivative of a portion of the signal, or a minimum value of the time derivative of a portion of the signal.

38. The computer-implemented method of claim 35 wherein an elementary-type physiological event is recognized as a sequence of input-signal characteristics.

39. The computer-implemented method of claim 35 wherein the recognition of elementary-type and primary-type events proceeds concurrently, and wherein the recognition of a next elementary-type event further proceeds in dependence on a partially-completed recognition of a current primary-type event.

40. The computer-implemented method of claim 35 wherein the recognition of a primary-type event proceeds in dependence on a pattern of elementary-type events, the pattern comprising specification of elementary-type events with pre-determined characteristics and order.

41. The computer-implemented method of claim 40 wherein the recognition of elementary-type and primary-type events proceeds concurrently, and wherein the recognition of a next elementary-type event further proceeds in dependence on at least one expected next elementary-type event that is determined by the pattern for the partially-recognized current primary-type event.

42. The computer-implemented method of claim 40 wherein the pattern of elementary-type events is specified by a regular expression.

43. The computer-implemented method of claim 40 wherein the pattern of elementary-type events is recognized by a finite state machine.

44. The computer-implemented method of claim 35 wherein creation of an elementary-type data-structure comprises providing identification of the portion of the input-signal defining the represented elementary-type event.

45. The computer-implemented method of claim 35 wherein creation of a primary-type data-structure comprises providing identification of the elementary-type data-structure instances representing the defining elementary-type events.

46. The computer-implemented method of claim 35 further comprising:
searching part or all of the created data-structure instances for one or more data-structure instances that satisfy a pre-determined physiological criterion, and
creating at least one instance of a view-type data-structure representing at least one data-structure instance found in search.

47. The computer-implemented method of claim 46 wherein the pre-determined physiological criterion comprises a pattern specifying data-structure instances with pre-determined characteristics and order.

48. The computer-implemented method of claim 47 wherein the pattern is recognized by a finite state machine.

49. The computer-implemented method of claim 46 wherein the pre-determined physiological criterion defines one or more of a period of apnea, a period of hypopnea, a cough, or a period of Cheyne-Stokes respiration.

50. A computer-implemented method for analyzing input signals from a monitored subject, the input signals reflecting physiological events in the subject generated by at least a first and a second physiological process, the method comprising:
recognizing at least one primary-type physiological event of the first process defined in dependence on one or more corresponding elementary-type physiological events, wherein the elementary-type events are defined in dependence on portions of the input signal,
creating at least one instance of a first-process data-structure representing characteristics of the recognized primary-type event
recognizing at least one primary-type physiological event of the second process defined in dependence on one or more corresponding elementary-type physiological events, wherein the elementary-type events are defined in dependence on portions of the input signal,
creating at least one instance of a second-process data-structure representing characteristics of the recognized primary-type event
wherein at least one primary-type data-structure instance comprises data identifying at least one other primary-type data-structure instance, the primary-type data-structure instances representing primary-type events of the first and second processes that are physiologically associated.

51. The computer-implemented method of claim 50 wherein the first and second physiological processes comprise a respiratory process, or a cardiac process, or a pulse oximetry process, or a capnometery process, or an electroencephalographic process, or an electrooculographic process, or a electromyographic process, or a sound microphone signal process, or a body temperature signal process, or an accelerometer signal process, or a blood glucose concentration signal process, or a blood pressure process, or a blood flow process.

52. The computer-implemented method of claim 50 wherein the physiological processes comprise a breath process, wherein primary-type physiological event are breaths and the elementary-type physiological events are breath phases.

53. The computer-implemented method of claim 52 wherein the breath phases comprise one or more of a begin inspiration phase, or a begin inspiratory flow phase, or a peak inspiratory flow phase, or a peak phase, or a begin expiratory flow phase, or a peak expiratory flow phase, or an end expiration phase.

54. The computer-implemented method of claim 50 wherein the physiological processes comprise a cardiac process, wherein the primary-type physiological event are heartbeats and the elementary-type physiological events are cardiac phases

55. The memory of claim 54 wherein the cardiac phases comprise a depolarization phase, or a repolarization phase, or an ECG P-wave phase, or an ECG QRS-complex phase, or an ECG T-wave phase, or a diastole phases or a systole phases.

56. The memory of claim 54 wherein the primary-type data-structures comprise indicia of ventricular volume or of ventricular wall motion.

57. The computer-implemented method of claim 50 further comprising:
searching part or all of the created data-structure instances for one or more data-structure instances that satisfy a pre-determined physiological criterion, and
creating at least one instance of a view-type data-structure representing at least one data-structure instance found in search.

58. The computer-implemented method of claim 57 wherein the pre-determined physiological criterion comprises specification of a pattern comprising data-structure instances.

59. The computer-implemented method of claim 50 further comprising:
searching part or all of the created data-structure instances for instances representing events generated by one of the physiological processes that satisfy a pre-determined physiological criterion, and
creating at least one instance of a view-type data-structure representing
(i) at least one data-structure instance found in search, and
(ii) at least one data-structure instance representing an event generated by the other of the physiological processes that is physiologically associated with the instances of (i).

60. The computer-implemented method of claim 59 wherein the data-structure instances of the first and second physiological processes are physiologically associated if the represented events have overlapping occurrence times.

61. The computer-implemented method of claim 60 wherein the primary-type data-structures represent heartbeats and breaths.

62. The computer-implemented method of claim 59 wherein the primary-type data-structures represent respiratory events, or cardiac events, or arterial pulse events, or capnometery process events, or electroencephalographic process events, or electrooculographic process events, or electromyographic process events, or sound microphone signal process events, or body temperature events, or accelerometer signal events, or blood glucose concentration events, or blood pressure events, or blood flow events.

63. The computer-implemented memory of claim 59 wherein the data-structure instances of the first and second physiological processes are associated if the represented events have occurrence times displaced by a determined amount.

64. The computer-implemented memory of claim 59 wherein the data-structure instances of the first and second physiological processes are physiologically associated if the represented events have a cause-and-effect relationship.

65. The computer-implemented method of claim 59 wherein the data-structure instances of the first and second physiological processes are manually associated by a user.

66. The computer-implemented method of claim 59 wherein the primary-type data-structures represent heartbeats and breaths, and wherein heartbeats and breaths are physiologically associated if their occurrence times overlap.

67. The computer-implemented method of claim 59 the primary-type data-structures represent heartbeats and arterial pulses, and wherein arterial pulses and heartbeats are physiologically associated if the arterial pulses result from the heartbeats.

68. The computer-implemented method of claim 59 wherein the primary-type data-structures represent breaths and arterial pulses, and wherein breaths and arterial pulses are physiologically associated if the oxygen saturation of the arterial pulses results from the breaths.

69. The computer-implemented method of claim 59 wherein the primary-type data-structures represent heartbeats and breaths, and wherein heartbeats and breaths are physiologically associated if the heartbeats are temporally displaced from the breaths by a displacement interval characteristic of an anxiety reaction.

70. A computer-implemented method for creating view-type data structures comprising:
in a computer-readable memory structured according to claim 1, searching part or all of the data-structure instances for data-structure instances that satisfy a pre-determined physiological criterion, and
creating at least one instance of a view-type data-structure representing at least one data-structure instance found in search.

71. The computer-implemented method of claim 70 further comprising accessing the data-structure instances represented by at least one created view-type data-structure instance.

72. The computer-implemented method of claim 70 wherein the physiological criterion specifies characteristics of individual data-structure instances.

73. The computer-implemented method of claim 70 wherein the physiological criterion specifies a pattern comprising a plurality of data-structure instances with pre-determined characteristics and relative order.

74. A computer system comprising:
a processor, and
a memory operatively coupled to the processor, wherein the memory comprises executable instructions for causing the processor to perform the method of claim 35 for analyzing input signals from a monitored subject.

75. The computer system of claim 74 further comprising user interface equipment.

76. The computer system of claim 74 further comprising communication equipment for the receiving input signals.

77. The computer system of claim 76 wherein the communication equipment comprises Internet access devices.

78. The computer system of claim 74 further comprising equipment for reading the input signals from a removable computer-readable media.

79. The computer system of claim 78 wherein the removable computer-readable media include memory cards.

80. The computer system of claim 74 wherein the memory further comprises executable instruction for causing the computer to perform the method of claim 46 for creating view-type data structures.

81. A computer system comprising:
a processor, and
a memory operatively coupled to the processor, wherein the memory comprises executable instructions for causing the processor to perform the method of claim 50 for analyzing input signals from a monitored subject.

82. A program product comprising a computer readable medium having instructions for causing a computer to execute the method of claim 35 for analyzing input signals from a monitored subject.

83. A program product comprising a computer readable medium having instructions for causing a computer to execute the method of claim 50 for analyzing input signals from a monitored subject.
